(19) United States
(12) Patent Application Publication
(10) Pub. No.: US 2020/0360500 A1
(43) Pub. Date: Nov. 19, 2020

(54) PNEUMOCOCCAL CONJUGATE VACCINE FORMULATIONS

(71) Applicants: Ramesh V. Chintala, West Point, PA (US); Akhilesh Bhambhani, West Point, PA (US); Christopher David Mensch, West Point, PA (US); Denise K. Nawrocki, West Point, PA (US); Jeffrey Thomas Blue, West Point, PA (US); Merck Sharp & Dohme Corp., Rahway, NJ (US)

(72) Inventors: Ramesh V. Chintala, Chalfont, PA (US); Akhilesh Bhambhani, Doylestown, PA (US); Christopher David Mensch, Lansdale, PA (US); Denise K. Nawrocki, Annandale, NJ (US); Jeffrey Thomas Blue, Telford, PA (US)

(73) Assignee: Merck Sharp & Dohme Corp., Rahway, NJ (US)

(21) Appl. No.: 16/638,846

(22) PCT Filed: Aug. 13, 2018
(86) PCT No.: PCT/US2018/046411

§ 371 Date: Feb. 13, 2020

Related U.S. Application Data
(60) Provisional application No. 62/546,428, filed on Aug. 16, 2017.

(51) Int. Cl. A61K 39/09; A61K 47/26; A61K 47/38; A61K 47/10; A61K 47/02

(52) U.S. Cl. CPC A61K 39/092; A61K 47/26; A61K 2039/55505; A61K 47/10; A61K 47/02; A61K 47/38

(57) ABSTRACT

The present invention provides polysaccharide-protein conjugate vaccine formulations comprising a buffer, surfactant, sugar, alkali or alkaline salt, aluminum adjuvant, optionally a bulking agent, and optionally a polymer.

### FIELD OF INVENTION

The present invention provides pneumococcal conjugate vaccine formulations comprising a buffer, surfactant, sugar, alkali or alkaline salt, aluminum adjuvant, optionally a bulking agent, and optionally a polymer.

### BACKGROUND OF THE INVENTION

Streptococcus pneumoniae, one example of an encapsulated bacterium, is a significant cause of serious disease world-wide. In 1997, the Centers for Disease Control and Prevention (CDC) estimated there were 3,000 cases of pneumococcal meningitis, 50,000 cases of pneumococcal bacteremia, 7,000,000 cases of pneumococcal otitis media and 500,000 cases of pneumococcal pneumonia annually in the United States. See Centers for Disease Control and Prevention, MMWR Morb Mortal Wkly Rep 1997, 46(RR-8):1-13. Furthermore, the complications of these diseases can be significant with some studies reporting up to 8% mortality and 25% neurologic sequelae with pneumococcal meningitis. See Arditi et al., 1998, Pediatrics 102:1087-97.

The multivalent pneumococcal polysaccharide vaccines that have been licensed for many years have proved invaluable in preventing pneumococcal disease in adults, particularly, the elderly and those at high-risk. However, infants and young children respond poorly to unconjugated pneumococcal polysaccharides. Bacterial polysaccharides are T-cell-independent immunogens, eliciting weak or no response in infants. Chemical conjugation of a bacterial polysaccharide immunogen to a carrier protein converts the immune response to a T-cell-dependent one in infants. Diphtheria toxoid (DTx, a chemically detoxified version of DT) and CRM₁₉₇ have been described as carrier proteins for bacterial polysaccharide immunogens due to the presence of T-cell-stimulating epitopes in their amino acid sequences.

The pneumococcal conjugate vaccine, Prevnar®, containing the 7 most frequently isolated serotypes (4, 6B, 9V, 14, 18C, 19F and 23F) causing invasive pneumococcal disease in young children and infants at the time, was first licensed in the United States in February 2000.

Prevnar 13® is a 13-valent pneumococcal polysaccharide-protein conjugate vaccine including serotypes 1, 3, 4, 5, 6A, 6B, 7F, 9V, 14, 18C, 19A, 19F and 23F. See, e.g., U.S. Patent Application Publication No. US 2006/0228380 A1, Prymula et al., 2006, Lancet 367:740-48 and Kieninger et al., Safety and Immunologic Non-inferiority of 13-valent Pneumococcal Conjugate Vaccine Compared to 7-valent Pneumococcal Conjugate Vaccine Given as a 4-Dose Series in Healthy Infants and Toddlers, presented at the 48^{th} Annual ICAAC/ISDA 46^{th} Annual Meeting, Washington D.C., Oct. 25-28, 2008. See, also, Dagan et al., 1998, Infect Immun. 66: 2093-2098 and Fattom, 1999, Vaccine 17:126.

Chinese Patent Application Publication No. CN 101590224 A describes a 14-valent pneumococcal polysaccharide-protein conjugate vaccine including serotypes 1, 2, 4, 5, 6A, 6B, 7F, 9N, 9V, 14, 18C, 19A, 19F and 23F.

U.S. Pat. No. 8,192,746 describes a 15-valent pneumococcal polysaccharide-protein conjugate vaccine having serotypes 1, 3, 4, 5, 6A, 6B, 7F, 9V, 14, 18C, 19A, 19F, 22F, 23F and 33F, all individually conjugated to CRM₁₉₇ polypeptides.

Multiple carrier protein systems have also been described. See e.g., U.S. Patent Application Publication Nos. 20100209450, 20100074922, 20090017059, 20090010959 and 20090017072.

Formulations comprising S. pneumoniae polysaccharide-protein conjugates and surfactants including polysorbate 80 (PS-80) and poloxamer 188 (P188) have been disclosed. See U.S. Pat. No. 8,562,999 and U.S. Patent Application Publication No. US20130273098, respectively.

### SUMMARY OF THE INVENTION

The present invention provides a formulation comprising (i) one or more polysaccharide-protein conjugates; (ii) a buffer having a pH in the range from about 5.0 to 7.5; (ii) an alkali or alkaline salt selected from the group consisting of magnesium chloride, calcium chloride, potassium chloride, sodium chloride or a combination thereof; (iii) a surfactant; (iv) a sugar selected from the group consisting of sucrose, trehalose and raffinose; optionally (v) a bulking agent; and optionally (vi) a polymer selected from the group consisting of carboxymethyl cellulose (CMC), hydroxypropyl cellulose (HPC), hydroxypropyl methylcellulose (HPMC), 2-hydroxyethyl cellulose (2-HEC), crosscarmellose, methyl cellulose, glycerol, polyethylene oxide, polyethylene glycol (PEG) and propylene glycol (PG), or a combination thereof and (vii) an aluminum adjuvant.

In one embodiment, the total concentration of sugar and bulking agent is at least about 50 mg/ml. In another embodiment, the total concentration of sugar and bulking agent is at least about 90 mg/ml. In a further embodiment, the total concentration of sugar and bulking agent is about 50-400 mg/ml, and the bulking agent to sugar ratio is greater than or equal to 1. In a further embodiment, the total concentration of sugar and bulking agent is about 50-150 mg/ml, and the bulking agent to sugar ratio is about 2:1. In one embodiment, the bulking agent is mannitol, glycine or lactose. In a further embodiment, the sugar is trehalose or sucrose.

In one embodiment, the polymer is carboxymethyl cellulose (CMC), hydroxypropyl cellulose (HPC), 2-hydroxyethyl cellulose (2-HEC), polyethylene glycol (PEG) or propylene glycol (PG) at about 1-25 mg/ml, or a combination thereof. In a preferred embodiment, the polymer is carboxymethyl cellulose (CMC). In one embodiment, the polysaccharide-protein concentration is 2-704, 5-500 or 4-92 μg/ml. In another embodiment, the polysaccharide-protein concentration is 40-80 or 4-92 μg/ml. In certain embodiments, the formulation comprises 0.1-0.5 mg/mL of Aluminum Phosphate Adjuvant (APA).

In certain embodiments, the surfactant is a poloxamer which has a molecular weight in the range from 1100 Da to 17,400 Da, 7,500 Da to 15,000 Da, or 7,500 Da to 10,000 Da. The poloxamer can be poloxamer 188 or poloxamer 407. In certain aspects, final concentration of the poloxamer is from 0.001 to 50 mg/ml, from 0.25 to 10 mg/ml. In certain embodiments, the surfactant is polysorbate 20. In certain aspects, the final concentration of the polysorbate 20 is in the range from 0.01 to 100 mg/ml, or from 0.25 to 1 mg/ml, or from 0.25 to 5 mg/ml.

In certain embodiments, the pH buffered saline solution can have a pH in the range from 5.0 to 7.5. The buffer can be selected from the group consisting of phosphate, succinate, L-histidine, MES, MOPS, HEPES, acetate or citrate. In one aspect, the buffer is L-histidine at a final concentration of 5 mM to 50 mM, or succinate at a final concentration of 1 mM to 10 mM. In a specific aspect, the L-histidine is at a final concentration of 20 mM±2 mM. The salt in the pH buffered saline solution can be magnesium chloride, potassium chloride, sodium chloride or a combination thereof. In one aspect, the pH buffered saline solution is sodium chloride. The saline can be present at a concentration from 20 mM to 170 mM.

In certain embodiments, the polysaccharide-protein conjugates comprise one or more pneumococcal polysaccharides conjugated to a carrier protein. In certain aspects, the carrier protein is selected from CRM₁₉₇, diphtheria toxin fragment B (DTFB), DTFB C8, Diphtheria toxoid (DT), tetanus toxoid (TT), fragment C of TT, pertussis toxoid, cholera toxoid, meningococcal outer membrane protein (OMPC), E. coli LT (heat-labile enterotoxin), E. coli ST (heat-stable enterotoxin), exotoxin A from Pseudomonas aeruginosa, and combinations thereof. In one specific aspect, one or more of the polysaccharide-protein conjugates are conjugated to CRM₁₉₇. In certain aspects, one or more of the polysaccharide protein conjugates is prepared using reductive amination in either an aqueous solvent or in a non-aqueous solvent such as dimethysufloxide (DMSO). In certain aspects, polysaccharide protein conjugates from serotypes 6A, 6B, 7F, 18C, 19A, 19F, and 23F can be prepared using reductive amination in DMSO, and polysaccharide protein conjugates from serotypes 1, 3, 4, 5, 9V, 14, 22F, and 33F can be prepared using reductive amination in aqueous solution. In certain aspects, each dose is formulated to contain: 4 μg/mL or 8 μg/mL of each saccharide, except for 6B at 8 μg/mL or 16 μg/mL; and about 64 μg/mL or 128 μg/mL CRM₁₉₇ carrier protein.

The present invention is also directed to a pneumococcal conjugate formulation comprising S. pneumoniae polysaccharides from serotypes 1, 3, 4, 5, 6A, 6B, 7F, 9V, 14, 18C, 19A, 19F, 22F, 23F and 33F conjugated to a CRM₁₉₇ polypeptide or capsular polysaccharides from at least one of serotypes 1, 2, 3, 4, 5, 6A, 6B, 6C, 6D, 6E, 6G, 6H, 7F, 7A, 7B, 7C, 8, 9A, 9L, 9N, 9V, 10F, 10A, 10B, 10C, 11F, 11A, 11B, 11C, 11D, 11E, 12F, 12A, 12B, 13, 14, 15F, 15A, 15B, 15C, 16F, 16A, 17F, 17A, 18F, 18A, 18B, 18C, 19F, 19A, 19B, 19C, 20A, 20B, 21, 22F, 22A, 23F, 23A, 23B, 24F, 24A, 24B, 25F, 25A, 27, 28F, 28A, 29, 31, 32F, 32A, 33F, 33A, 33B, 33C, 33D, 33E, 34, 35F, 35A, 35B, 35C, 36, 37, 38, 39, 40, 41F, 41A, 42, 43, 44, 45, 46, 47F, 47A, 48, CWPS1, CWPS2, CWPS3 of Streptococcus pneumoniae conjugated to CRM₁₉₇, containing 4 μg/mL or 8 μg/mL of each saccharide, except for 6B at 8 μg/mL or 16 g/mL; and about 64 μg/mL or 128 μg/mL CRM₁₉₇ carrier protein; a pH buffered saline solution having a pH in the range from about 5.0 to 7.5, about 150 mM NaCl, about 2 mg/ml Polysorbate 20, 250 μg/ml APA, with about 50 mg/ml mannitol, and about 20 mg/ml sucrose; about 60 mg/ml mannitol, about 40 mg/ml sucrose; about 90 mg/ml sucrose, about 5 mg/ml CMC; about 90 mg/ml sucrose, about 5 mg/ml 2-HEC; about 90 mg/ml sucrose, about 5 mg/ml HPC; about 90 mg/ml sucrose, about 5 mg/ml CMC, and about 5 mg/ml PG; about 40 mg/ml sucrose, about 60 mg/ml mannitol, and about 5 mg/ml CMC; or about 40 mg/ml sucrose, about 60 mg/ml mannitol, about 5 mg/ml CMC and about 5 mg/ml PG. In certain aspects, the buffer is histidine.

In another aspect, the invention provides a vaccine formulation comprising a 15-valent pneumococcal conjugate (15vPnC) consisting essentially of S. pneumoniae polysaccharide from serotypes 1, 3, 4, 5, 6A, 6B, 7F, 9V, 14, 18C, 19A, 19F, 22F, 23 F and 33F conjugated to CRM₁₉₇ at about 20-150, 2-704 or 4-92 μg/ml, a pH buffered saline solution having a pH in the range from about 5.0 to 7.5, about 30-150 mM NaCl, about 0.05-2 mg/ml Polysorbate 20, about 20-250 mg/ml sucrose, about 30-100 mg/ml mannitol, about 0.1-0.75 mg/ml APA, about 1-10 mg/ml CMC and optionally about 1-10 mg/ml PG.

In another aspect, the invention provides a vaccine formulation comprising polysaccharides from at least one of serotypes 1, 2, 3, 4, 5, 6A, 6B, 6C, 6D, 6E, 6G, 6H, 7F, 7A, 7B, 7C, 8, 9A, 9L, 9N, 9V, 10F, 10A, 10B, 10C, 11F, 11A, 11B, 11C, 11D, 11E, 12F, 12A, 12B, 13, 14, 15F, 15A, 15B, 15C, 16F, 16A, 17F, 17A, 18F, 18A, 18B, 18C, 19F, 19A, 19B, 19C, 20A, 20B, 21, 22F, 22A, 23F, 23A, 23B, 24F, 24A, 24B, 25F, 25A, 27, 28F, 28A, 29, 31, 32F, 32A, 33F, 33A, 33B, 33C, 33D, 33E, 34, 35F, 35A, 35B, 35C, 36, 37, 38, 39, 40, 41F, 41A, 42, 43, 44, 45, 46, 47F, 47A, 48, CWPS1, CWPS2, CWPS3 of Streptococcus pneumoniae conjugated to CRM₁₉₇ at about 20-150, 4-92 or 2-704 μg/ml, a pH buffered saline solution having a pH in the range from about 5.0 to 7.5, about 30-150 mM NaCl, about 0.05-2 mg/ml Polysorbate 20, about 20-250 mg/ml sucrose, about 30-100 mg/ml mannitol, about 0.1-0.75 mg/ml APA, about 1-10 mg/ml CMC and optionally about 1-10 mg/ml PG.

In another aspect, the invention provides a container comprising a pneumococcal conjugate vaccine comprising 4-704 or 4-92 μg S. pneumoniae polysaccharide from serotypes 1, 3, 4, 5, 6A, 6B, 7F, 9V, 14, 18C, 19A, 19F, 22F, 23 F and 33F conjugated to CRM₁₉₇ or a pneumococcal conjugate vaccine comprising capsular polysaccharides from at least one of serotypes 1, 2, 3, 4, 5, 6A, 6B, 6C, 6D, 6E, 6G, 6H, 7F, 7A, 7B, 7C, 8, 9A, 9L, 9N, 9V, 1° F., 10A, 10B, 10C, 11F, 11A, 11B, 11C, 11D, 11E, 12F, 12A, 12B, 13, 14, 15F, 15A, 15B, 15C, 16F, 16A, 17F, 17A, 18F, 18A, 18B, 18C, 19F, 19A, 19B, 19C, 20A, 20B, 21, 22F, 22A, 23F, 23A, 23B, 24F, 24A, 24B, 25F, 25A, 27, 28F, 28A, 29, 31, 32F, 32A, 33F, 33A, 33B, 33C, 33D, 33E, 34, 35F, 35A, 35B, 35C, 36, 37, 38, 39, 40, 41F, 41A, 42, 43, 44, 45, 46, 47F, 47A, 48, CWPS1, CWPS2, CWPS3 of Streptococcus pneumoniae conjugated to CRM₁₉₇ and a formulation selected from the group consisting of: about 3.5 mg CMC, about 62 mg sucrose, about 0.18 mg APA, about 1.4 mg PS20, about 6.3 mg NaCl, about 2.2 mg Histidine; about 3.5 mg CMC, about 3.5 mg PG, about 63 mg sucrose, about 0.18 mg APA, about 1.4 mg PS20, about 6.3 mg NaCl, about 2.2 mg Histidine; about 3.5 mg CMC, about 28 mg sucrose, 42 mg mannitol, about 0.18 mg APA, about 1.4 mg PS20, about 6.3 mg NaCl, about 2.2 mg Histidine; about 3.5 mg CMC, and about 3.5 mg PG, about 28 mg sucrose, about 42 mg mannitol, about 0.18 mg APA, about 1.4 mg PS20, about 2.1 mg NaCl, about 2.2 mg Histidine. In one embodiment, the vaccine has d(0.50) less than 15 μm or less than 10 m.

The present invention also provides a method of obtaining a dried conjugated vaccine preabsorbed on an aluminum adjuvant, through the application of microwave radiation in a traveling wave format in a vacuum chamber to obtain dried lyosphere and/or cakes with no visible sign of boiling.

### DETAILED DESCRIPTION OF THE INVENTION

The present invention is based, in part, on the discovery that sugars and bulking agents in conjugate vaccine formulations with an aluminum adjuvant can decrease their tendency to aggregate during lyophilization, microwave drying and lyosphere formation.

The term “about”, when modifying the quantity (e.g., mM, or M) of a substance or composition, the percentage (v/v or w/v) of a formulation component, the pH of a solution/formulation, or the value of a parameter characterizing a step in a method, or the like refers to variation in the numerical quantity that can occur, for example, through typical measuring, handling and sampling procedures involved in the preparation, characterization and/or use of the substance or composition; through inadvertent error in these procedures; through differences in the manufacture, source, or purity of the ingredients employed to make or use the compositions or carry out the procedures; and the like. In certain embodiments, “about” can mean a variation of ±0.1%, 0.5%, 1%, 2%, 3%, 4%, 5%, or 10%.

As used herein, the term “polysaccharide” (Ps) is meant to include any antigenic saccharide element (or antigenic unit) commonly used in the immunologic and bacterial vaccine arts, including, but not limited to, a “saccharide”, an “oligosaccharide”, a “polysaccharide”, a “liposaccharide”, a “lipo-oligosaccharide (LOS)”, a “lipopolysaccharide (LPS)”, a “glycosylate”, a “glycoconjugate” and the like.

As used herein, the term “comprises” when used with the immunogenic composition of the invention refers to the inclusion of any other components (subject to limitations of “consisting of” language for the antigen mixture), such as adjuvants and excipients. The term “consisting of” when used with the multivalent polysaccharide-protein conjugate mixture refers to a mixture having those particular S. pneumoniae polysaccharide protein conjugates and no other S. pneumoniae polysaccharide protein conjugates from a different serotype.

The term “bulking agents” comprise agents that provide the structure of the freeze-dried product. Common examples used for bulking agents include mannitol, glycine, and lactose. In addition to providing a pharmaceutically elegant cake, bulking agents may also impart useful qualities in regard to modifying the collapse temperature, providing freeze-thaw protection, moisture reduction and enhancing the protein stability over long-term storage. These agents can also serve as tonicity modifiers.

As defined herein, the terms “precipitation”, “precipitate”, “particulate formation”, “agglomeration”, “clouding”, and “aggregation” may be used interchangeably and are meant to refer to any physical interaction or chemical reaction which results in the agglomeration of a polysaccharide-protein conjugate. The process of aggregation (e.g., protein aggregation) may be induced by numerous physicochemical stresses, including heat, pressure, pH, agitation, shear forces, freeze-thawing, dehydration, heavy metals, phenolic compounds, silicon oil, denaturants and the like.

The terms “lyophilization,” “lyophilized,” and “freeze-dried” refer to a process by which the material to be dried is first frozen and then the ice or frozen solvent is removed by sublimation in a vacuum environment. An excipient may be included in pre-lyophilized formulations to enhance stability of the lyophilized product upon storage.

“Lyosphere,” as used herein, refers to dried frozen unitary bodies comprising a therapeutically active agent which are substantially spherical or ovoid-shape. In some embodiments, the lyosphere diameter is from about 2 to about 12 mm, preferably from 2 to 8 mm, such as from 2.5 to 6 mm or 2.5 to 5 mm. In some embodiments, the volume of the lyosphere is from about 20 to 550 μL, preferably from 20 to 100 μL, such as from 20 to 50 L. In embodiments wherein the lyosphere is not substantially spherical, the size of the lyosphere can be described with respect to its aspect ratio, which is the ratio of the longer dimension to the shorter dimension. The aspect ratio of the lyospheres can from 0.5 to 2.5, preferably from 0.75 to 2, such as from 1 to 1.5. Lyospheres can be made, for example, by loading an aliquot of liquid in the form of a droplet (e.g., about 20, 50, 100 or 250 microliters) onto a solid, flat surface in such a way that the droplet remains intact. In an embodiment of the invention, the surface is a plate, e.g., a metal plate, e.g. at a temperature of about −180° C. to about −196° C. or about −180° C. to about −273° C. For example, in an embodiment of the invention, the liquid is loaded onto the surface by way of a dispensing tip. In an embodiment of the invention, the liquid is dispensed at a dispensing speed of about 3 ml/min to about 75 ml/min, about 5 ml/min to about 75 ml/min; about 3 ml/min to about 60 ml/min, about 20 ml/min to about 75 ml/min; and about 20 ml/min to about 60 ml/min. In an embodiment of the invention, the aliquot that is dispensed is 250 microliters and the dispensing speed is between about 5 ml/min to about 75 ml/min, or wherein the aliquot is 100 microliters and the dispensing speed is between about 3 ml/min to about 60 ml/min. In an embodiment of the invention, the gap between a dispensing tip and the surface onto which the liquid is dispensed if about 0.1 cm or more (e.g., about 0.5 cm or between 0.1 cm and 1 cm or between 0.1 cm and 0.75 cm). Once on the surface, the droplet is frozen and then subjected to drying. Methods for making lyospheres are known in the art. See e.g., U.S. Pat. No. 5,656,597; WO2013066769; WO2014093206; WO2015057540; WO2015057541 or WO2015057548.

A “reconstituted” formulation is one that has been prepared by dissolving dried vaccine formulation in a diluent such that the vaccine is dispersed in the reconstituted formulation. The reconstituted formulation is suitable for administration, (e.g. intramuscular administration), and may optionally be suitable for subcutaneous administration.

As defined herein, a “surfactant” of the present invention is any molecule or compound that lowers the surface tension of an immunogenic composition formulation. A “surfactant system” comprises a surfactant but may allow for the inclusion of additional excipients such as polyols that increase the effects of the surfactant.

As used herein, “x % (w/v)” is equivalent to x g/100 ml (for example 0.2% w/v PS20 equals 2 mg/ml PS20).

“Microwave Vacuum Drying” as used herein, refers to a drying method that utilizes microwave radiation (also known as radiant energy or non-ionizing radiation) for the formation of dried vaccine products (preferably, <6% moisture) of a vaccine formulation through sublimation. In certain embodiments, the microwave drying is performed as described in US2016/0228532.

An immunogenic composition of the invention may be a multivalent composition containing one or more antigens conjugated to one or more carrier proteins. In certain embodiments of the invention, the antigen is a saccharide from an encapsulated bacteria. In such vaccines, the saccharides are composed of long chains of sugar molecules that resemble the surface of certain types of bacteria. Encapsulated bacteria include, but are not limited to, Streptococcus pneumoniae, Neisseria meningitides and Haemophilus influenzae type b. In other embodiments, the polysaccharide is from Salmonella typhi, Salmonella paratyphi A, Salmonella typhimurium, Escherichia coli O157, Vibrio cholerae O1 and O139. The antigens may be from the same organism or may be from different organisms. In preferred embodiments of the invention, the antigens are Streptococcus pneumoniae capsular polysaccharides.

In embodiments where two carrier proteins are used, each capsular polysaccharide not conjugated to the first carrier protein is conjugated to the same second carrier protein (e.g., each capsular polysaccharide molecule being conjugated to a single carrier protein). In another embodiment, the capsular polysaccharides not conjugated to the first carrier protein are conjugated to two or more carrier proteins (each capsular polysaccharide molecule being conjugated to a single carrier protein). In such embodiments, each capsular polysaccharide of the same serotype is typically conjugated to the same carrier protein.

Diphtheria Toxin, an exotoxin secreted by Corynebacterium diphtheriae, is a classic A-B toxin composed of two subunits (fragments) linked by disulfide bridges and having three domains. Fragment A (DTFA) contains the ADP-ribose catalytic C domain, while Fragment B (DTFB) contains the central translocation T domain and a carboxy terminal receptor-binding R domain. DTFB is the non-toxic moiety constituting approximately 60% of the total amino acid sequence of DT. See e.g., Gill, D. M. and Dinius, L. L., J. Biol. Chem., 246, 1485-1491 (1971), Gill, D. M. and Pappenheimer, Jr., A. M., J. Biol. Chem., 246, 1492-1495 (1971), Collier, R. J. and Kandel, J., J. Biol. Chem., 246, 1496-1503 (1971); and Drazin, R., Kandel, J., and Collier, R. J., J. Biol. Chem., 246, 1504-1510 (1971).

The completed amino acid sequence of Diphtheria Toxin has been published. See Greenfield, L., Bjorn, M. J., Horn, G., Fong, D., Buck, G. A., Collier, R. J. and Kaplan, D. A., Proc. Nat. Acad. Sci. USA 80, 6853-6857 (1983). Specifically, DTFB comprises amino acid residues 194 to 535 of DT.

The CRM₁₉₇ carrier protein is a mutant form of DT that is rendered non-toxic by a single amino acid substitution in Fragment A at residue 52. CRM₁₉₇ and DT share complete sequence homology in Fragment B. Major T-cell epitopes were found predominantly in the B fragment of the DT amino acid sequence. See Bixler et al., Adv Exp Med Biol. (1989) 251:175-80; Raju et al., Eur. J. Immunol. (1995) 25: 3207-3214; Diethelm-Okita et al., J Infect Dis (2000) 181:1001-9; and McCool et al., Infect. and Immun. 67 (September 1999), p. 4862-4869.

Use of DTFB as described herein includes diphtheria toxin deletions of the ADP-ribosylation activity domain. Use of DTFB also includes variants having at least 90%, 95% or 99% sequence identity including deletions, substitutions and additions. An example of a variant is a deletion or mutation of the Cysteine 201. DTFB (C8) means diphtheria toxin deleted of the ADP-ribosylatin activation domain, and with cysteine 201 removed or mutated. Use of DTFB also includes fragments that cover sequence 265-450 of DT, which includes the published T-cell epitopes (See Bixler et al., Adv Exp Med Biol. (1989) 251:175-80; Raju et al., Eur. J. Immunol. (1995) 25: 3207-3214). DTFB also includes states of monomer, dimer, or oligomers. Use of DTFB also includes any protein complex (excluding full length DT or CRM₁₉₇), hybrid proteins, or conjugated proteins that contain the DTFB or fragments. Use of DTFB also includes chemically modified DTFB or fragments (i.e. pegylation, unnatural amino acid modification).

In certain embodiments, the DTFB is produced from enzymatic digestion and reduction of the native DT or the mutant CRM₁₉₇ with subsequent purification by adsorptive chromatography. Thus, it is envisioned that a purified DTFB, with or without a mutation at the DT C201 residue, could be prepared similarly from full length native or C201-mutated DT or CRM₁₉₇, or from variants thereof in which the A-fragment is truncated. It is specifically known that multimodal resins marketed as Capto™ Adhere and Capto™ MMC and Tris concentrations in excess of 50 mM during the chromatography cycle provide exceptional modes of purifying the cleaved native DTFB.

In certain embodiments, the preparation of DTFB includes up to 10 mM DTT. DTT prevents dimerization caused by disulfide bond formation between DTFB monomers due to free cysteine at residue position 201. In such cases, nickel is not added to the conjugation reaction mixture. However, the conjugation reaction proceeds by the same method otherwise. In a case where DTT is not used, dimerized DTFB may be conjugated to Ps and nickel, in a preferred embodiment, would be added to sequester residual, inhibitory cyanide to improve the extent of conjugation.

The removal of free cysteine (mutation of the DT C201) in the DTFB is expected to give similar behavior in the multimodal resins. Removal of the free cysteine is expected to eliminate the need for DTT since dimerization by disulfide bond formation between free cysteine would not be feasible. Increasing the Tris buffer concentration and the sodium chloride elution buffer concentration has been demonstrated to improve the recovery of DTFB protein from the Capto MMC chromatography resin. It is expected that the DTFB purification can be achieved using other multimodal resins.

In certain embodiments, the DTFB is expressed recombinantly with or without the mutation of the DT C201 residue and subsequently purified by various techniques known to those skilled in the art.

In a particular embodiment of the present invention, CRM₁₉₇ is used as a carrier protein. CRM₁₉₇ is a non-toxic variant (i.e., toxoid) of diphtheria toxin. In one embodiment, it is isolated from cultures of Corynebacterium diphtheria strain C7 (P197) grown in casamino acids and yeast extract-based medium. In another embodiment, CRM₁₉₇ is prepared recombinantly in accordance with the methods described in U.S. Pat. No. 5,614,382. Typically, CRM₁₉₇ is purified through a combination of ultra-filtration, ammonium sulfate precipitation, and ion-exchange chromatography. In some embodiments, CRM₁₉₇ is prepared in Pseudomonas fluorescens using Pfenex Expression Technology™ (Pfenex Inc., San Diego, Calif.).

DTFB and variants thereof can be used as a carrier protein for antigens, including protein (peptides) and saccharides. Other suitable carrier proteins include additional inactivated bacterial toxins such as DT (Diphtheria toxoid), TT (tetanus toxid) or fragment C of TT, pertussis toxoid, cholera toxoid (e.g., as described in International Patent Application Publication No. WO 2004/083251), E. coli LT, E. coli ST, and exotoxin A from Pseudomonas aeruginosa. Bacterial outer membrane proteins such as outer membrane complex c (OMPC), porins, transferrin binding proteins, pneumococcal surface protein A (PspA; See International Application Patent Publication No. WO 02/091998), pneumococcal adhesin protein (PsaA), C5a peptidase from Group A or Group B streptococcus, or Haemophilus influenzae protein D, pneumococcal pneumolysin (Kuo et al., 1995, Infect Immun 63; 2706-13) including ply detoxified in some fashion for example dPLY-GMBS (See International Patent Application Publication No. WO 04/081515) or dPLY-formol, PhtX, including PhtA, PhtB, PhtD, PhtE and fusions of Pht proteins for example PhtDE fusions, PhtBE fusions (See International Patent Application Publication Nos. WO 01/98334 and WO 03/54007), can also be used. Other proteins, such as ovalbumin, keyhole limpet hemocyanin (KLH), bovine serum albumin (BSA) or purified protein derivative of tuberculin (PPD), PorB (from N. meningitidis), PD (Haemophilus influenzae protein D; see, e.g., European Patent No. EP 0 594 610 B), or immunologically functional equivalents thereof, synthetic peptides (See European Patent Nos. EP0378881 and EP0427347), heat shock proteins (See International Patent Application Publication Nos. WO 93/17712 and WO 94/03208), pertussis proteins (See International Patent Application Publication No. WO 98/58668 and European Patent No. EP0471177), cytokines, lymphokines, growth factors or hormones (See International Patent Application Publication No. WO 91/01146), artificial proteins comprising multiple human CD4+ T cell epitopes from various pathogen derived antigens (See Falugi et al., 2001, Eur J Immunol 31:3816-3824) such as N19 protein (See Baraldoi et al., 2004, Infect Immun 72:4884-7), iron uptake proteins (See International Patent Application Publication No. WO 01/72337), toxin A or B of C. difficile (See International Patent Publication No. WO 00/61761), and flagellin (See Ben-Yedidia et al., 1998, Immunol Lett 64:9) can also be used as carrier proteins.

Other DT mutants can be used as the second carrier protein, such as CRM₁₇₆, CRM₂₂₈, CRM₄₅ (Uchida et al., 1973, J Biol Chem 218:3838-3844); CRM₉, CRM₄₅, CRM₁₀₂, CRM₁₀₃ and CRM₁₀7 and other mutations described by Nicholls and Youle in Genetically Engineered Toxins, Ed: Frankel, Maecel Dekker Inc, 1992; deletion or mutation of Glu-148 to Asp, Gln or Ser and/or Ala 158 to Gly and other mutations disclosed in U.S. Pat. No. 4,709,017 or 4,950,740; mutation of at least one or more residues Lys 516, Lys 526, Phe 530 and/or Lys 534 and other mutations disclosed in U.S. Pat. No. 5,917,017 or 6,455,673; or fragment disclosed in U.S. Pat. No. 5,843,711. Such DT mutants can also be used to make DTFB variants where the variants comprise the B fragment containing the epitope regions.

In one embodiment, the present invention provides an immunogenic composition comprising polysaccharide-protein conjugates comprising capsular polysaccharides from at least one of serotypes 1, 2, 3, 4, 5, 6A, 6B, 6C, 6D, 6E, 6G, 6H, 7F, 7A, 7B, 7C, 8, 9A, 9L, 9N, 9V, 10F, 10A, 10B, 10C, 11F, 11A, 11B, 11C, 11D, 11E, 12F, 12A, 12B, 13, 14, 15F, 15A, 15B, 15C, 16F, 16A, 17F, 17A, 18F, 18A, 18B, 18C, 19F, 19A, 19B, 19C, 20A, 20B, 21, 22F, 22A, 23F, 23A, 23B, 24F, 24A, 24B, 25F, 25A, 27, 28F, 28A, 29, 31, 32F, 32A, 33F, 33A, 33B, 33C, 33D, 33E, 34, 35F, 35A, 35B, 35C, 36, 37, 38, 39, 40, 41F, 41A, 42, 43, 44, 45, 46, 47F, 47A, 48, CWPS1, CWPS2, CWPS3, of Streptococcus pneumoniae conjugated to one or more carrier proteins, and a pharmaceutically acceptable carrier. In one embodiment, the present invention provides an immunogenic composition comprising polysaccharide-protein conjugates comprising capsular polysaccharides from at least one of serotypes 1, 2, 3, 4, 5, 6A, 6B, 6C, 6D, 7B, 7C, 7F, 8, 9N, 9V, 10A, 11A, 12F, 14, 15A, 15B, 15C, 16F, 17F, 18C, 19A, 19F, 20, 21, 22A, 22F, 23A, 23B, 23F, 24F, 27, 28A, 31, 33F, 34, 35A, 35B, 35F, and 38 of Streptococcus pneumoniae conjugated to one or more carrier proteins, and a pharmaceutically acceptable carrier. In certain embodiments of the invention, the immunogenic composition comprises, consists essentially of, or consists of capsular polysaccharides from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 serotypes individually conjugated to CRM₁₉₇. In certain aspects of the invention, CRM₁₉₇ is the only carrier protein used. In other embodiments, the polysaccharide-protein conjugate formulation is a 13-valent pneumococcal conjugate (13vPnC) formulation consisting essentially of S. pneumoniae polysaccharide from serotypes 1, 3, 4, 5, 6A, 6B, 7F, 9V, 14, 18C, 19A, 19F, and 23 F conjugated to CRM₁₉₇. In further embodiments, the polysaccharide-protein conjugate formulation is a 10-valent pneumococcal conjugate (10 vPnC) formulation consisting essentially of S. pneumoniae polysaccharide from serotypes 1, 4, 5, 6B, 7F, 9V, 14, 18C, 19F, and 23F conjugated to Protein D from Non-Typeable Haemophilus influenzae.

In certain embodiments, the immunogenic compositions described above optionally further comprise capsular polysaccharides from one additional S. pneumoniae serotype selected from at least one of 1, 2, 3, 4, 5, 6A, 6B, 6C, 6D, 7B, 7C, 7F, 8, 9N, 9V, 10A, 11A, 12F, 14, 15A, 15B, 15C, 16F, 17F, 18C, 19A, 19F, 20, 21, 22A, 22F, 23A, 23B, 23F, 24F, 27, 28A, 31, 33F, 34, 35A, 35B, 35F, and 38 conjugated to a second carrier protein (which is distinct in at least one amino acid from the first carrier protein). Preferably, saccharides from a particular serotype are not conjugated to more than one carrier protein.

In certain embodiments of the invention, the immunogenic composition of the invention further comprises capsular polysaccharides from at least one additional serotype conjugated to a second carrier protein. In these embodiments, the immunogenic composition comprises, consists essentially of, or consists of capsular polysaccharides from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 36, 37, 38, 39, 40, 41, 42, 43, or 44 serotypes individually conjugated to a second carrier protein which is not CRM₁₉₇.

In certain embodiments of the invention, the immugenic composition comprises, consists essentially of, or consists of, capsular polysaccharides from N serotypes where N is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44; and capsular polysaccharides from each of the N serotypes are conjugated to the first protein carrier which is CRM₁₉₇. In other embodiments of the invention, capsular polysaccharides from 1, 2, 3 . . . or N-1 serotypes are conjugated to the first protein carrier, and capsular polysaccharides from N-1, N-2, N-3 . . . 1 serotypes are conjugated to the second protein carrier which is different from CRM₁₉₇.

In one specific embodiment of the invention, the present invention provides a 15-valent immunogenic composition comprising, consisting essentially of, or consisting of capsular polysaccharides from serotypes 1, 3, 4, 5, 6A, 6B, 7F, 9V, 14, 18C, 19A, 19F, 22F, 23F, and 33F conjugated to CRM₁₉₇.

Capsular polysaccharides from Streptococcus pneumoniae can be prepared by standard techniques known to those skilled in the art. For example, polysaccharides can be isolated from bacteria and may be sized to some degree by known methods (see, e.g., European Patent Nos. EP497524 and EP497525); and preferably by microfluidisation accomplished using a homogenizer or by chemical hydrolysis. In one embodiment, S. pneumoniae strains corresponding to each polysaccharide serotype are grown in a soy-based medium. The individual polysaccharides are then purified through standard steps including centrifugation, precipitation, and ultra-filtration. See, e.g., U.S. Patent Application Publication No. 2008/0286838 and U.S. Pat. No. 5,847,112. Polysaccharides can be sized in order to reduce viscosity and/or to improve filterability of subsequent conjugated products. In the present invention, capsular polysaccharides are prepared from one or more of serotypes 1, 2, 3, 4, 5, 6A, 6B, 6C, 6D, 7B, 7C, 7F, 8, 9N, 9V, 10A, 11A, 12F, 14, 15A, 15B, 15C, 16F, 17F, 18C, 19A, 19F, 20, 21, 22A, 22F, 23A, 23B, 23F, 24F, 27, 28A, 31, 33F, 34, 35A, 35B, 35F, and 38.

The purified polysaccharides are chemically activated to introduce functionalities capable of reacting with the carrier protein. Once activated, each capsular polysaccharide is separately conjugated to a carrier protein to form a glycoconjugate. The polysaccharide conjugates may be prepared by known coupling techniques.

In one embodiment, the chemical activation of the polysaccharides and subsequent conjugation to the carrier protein are achieved by means described in U.S. Pat. Nos. 4,365,170, 4,673,574 and 4,902,506. Briefly, the pneumococcal polysaccharide is reacted with a periodate-based oxidizing agent such as sodium periodate, potassium periodate, or periodic acid resulting in random oxidative cleavage of vicinal hydroxyl groups to generate reactive aldehyde groups.

Direct aminative coupling of the oxidized polysaccharide to primary amine groups on the protein carrier (mainly lysine residues) can be accomplished by reductive amination. For example, conjugation is carried out by reacting a mixture of the activated polysaccharide and carrier protein with a reducing agent such as sodium cyanoborohydride in the presence of nickel. The conjugation reaction may be carried out in aqueous solution or in an organic solvent such as dimethylsulfoxide (DMSO). See, e.g., US2015/0231270 A1, EP 0471 177 B1, US2011/0195086 A1. At the conclusion of the conjugation reaction, unreacted aldehydes are capped by addition of a strong reducing agent, such as sodium borohydride.

In one embodiment, prior to formulation, each pneumococcal capsular polysaccharide antigen is individually purified from S. pneumoniae, activated to form reactive aldehydes, and then covalently conjugated using reductive amination with sodium cyanoboroydride in the presence of nickel to the first or second carrier protein. Nickel complexes with residual, inhibitory cyanide from sodium cyanoborohydride reducing agent used for reductive amination.

In certain embodiments, the conjugation reaction is performed by reductive amination wherein nickel is used for greater conjugation reaction efficiency and to aid in free cyanide removal. Transition metals are known to form stable complexes with cyanide and are known to improve reductive methylation of protein amino groups and formaldehyde with sodium cyanoborohydride (Gidley et al., 1982, Biochem J. 203: 331-334; Jentoft et al., 1980, Anal Biochem. 106: 186-190). By complexing residual, inhibitory cyanide, the addition of nickel increases the consumption of protein during the conjugation of and leads to formation of larger, potentially more immunogenic conjugates.

Variability in free cyanide levels in commercial sodium cyanoborohydride reagent lots may lead to inconsistent conjugation performance, resulting in variable conjugate attributes, including molecular mass and polysaccharide-to-protein ratio. The addition of nickel to the conjugation reaction reduces the level of free cyanide and thus improves the degree of lot-to-lot conjugate consistency.

In another embodiment, the conjugation method may employ activation of polysaccharide with 1-cyano-4-dimethylamino pyridinium tetrafluoroborate (CDAP) to form a cyanate ester. The activated saccharide may be coupled directly to an amino group on the carrier protein.

In another embodiment, a reactive homobifunctional or heterobifunctional group may be introduced on the activated polysaccharide by reacting the cyanate ester with any of several available modalities. For example, cystamine or cysteamine may be used to prepare a thiolated polysaccharide which could be coupled to the carrier via a thioether linkage obtained after reaction with a maleimide-activated carrier protein (for example using GMBS) or a haloacetylated carrier protein (for example using iodoacetimide [e.g. ethyl iodoacetimide HCl] or N-succinimidyl bromoacetate or SIAB, or SIA, or SBAP). In a preferred embodiment, the cyanate ester is reacted with hexane diamine or adipic acid dihydrazide (ADH) and the resultant amino-derivatised saccharide is conjugated to a free carboxy group on the carrier protein using carbodiimide (e.g. EDAC or EDC) chemistry. Such conjugates are described in International Patent Application Publication Nos. WO 93/15760, WO 95/08348 and WO 96/29094; and Chu et al., 1983, Infect. Immunity 40:245-256.

Other suitable conjugation methods use carbodiimides, hydrazides, active esters, norborane, p-nitrobenzoic acid, N-hydroxysuccinimide, S-NHS, EDC, TSTU. Many are described in International Patent Application Publication No. WO 98/42721. Conjugation may involve a carbonyl linker which may be formed by reaction of a free hydroxyl group of the saccharide with CDI (See Bethell et al., 1979, J. Biol. Chem. 254:2572-4; Hearn et al., 1981, J. Chromatogr. 218:509-18) followed by reaction with carrier protein to form a carbamate linkage. This chemistry consists of reduction of the anomeric terminus of a carbohydrate to form a primary hydroxyl group followed by reaction of the primary hydroxyl with CDI to form a carbamate intermediate and subsequent coupling to protein carrier amino groups. The reaction may require optional protection/deprotection of other primary hydroxyl groups on the saccharide.

Following conjugation, the polysaccharide-protein conjugates are purified to remove excess conjugation reagents as well as residual free protein and free polysaccharide by one or more of any techniques well known to the skilled artisan, including concentration/diafiltration operations, ultrafiltration, precipitation/elution, column chromatography, and depth filtration. See, e.g., U.S. Pat. No. 6,146,902.

After the individual glycoconjugates are purified, they are compounded to formulate the immunogenic composition of the present invention. These pneumococcal conjugates are prepared by separate processes and bulk formulated into a single dosage formulation.

### Pharmaceutical/Vaccine Compositions

The present invention further provides compositions, including pharmaceutical, immunogenic and vaccine compositions, comprising, consisting essentially of, or alternatively, consisting of any of the polysaccharide serotype combinations described above together with a pharmaceutically acceptable carrier and an adjuvant. In one embodiment, the compositions comprise, consist essentially of, or consist of 2, 3, 4, 5, 6A, 6B, 6C, 6D, 6E, 6G, 6H, 7F, 7A, 7B, 7C, 8, 9A, 9L, 9N, 9V, 1° F., 10A, 10B, 10C, 11F, 11A, 11B, 11C, 11D, 11E, 12F, 12A, 12B, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 distinct polysaccharide-protein conjugates, wherein each of the conjugates contains a different capsular polysaccharide conjugated to either the first carrier protein or the second carrier protein, and wherein the capsular polysaccharides from at least one of serotypes 1, 2, 3, 4, 5, 6A, 6B, 6C, 6D, 7B, 7C, 7F, 8, 9N, 9V, 10A, 11A, 12F, 14, 15A, 15B, 15C, 16F, 17F, 18C, 19A, 19F, 20, 21, 22A, 22F, 23A, 23B, 23F, 24F, 27, 28A, 31, 33F, 34, 35A, 35B, 35F, and 38 of Streptococcus pneumoniae are conjugated to a first carrier protein selected from CRM₁₉₇, and optionally having additional S. pneumoniae serotypes selected from serotypes 1, 2, 3, 4, 5, 6A, 6B, 6C, 6D, 7B, 7C, 7F, 8, 9N, 9V, 10A, 11A, 12F, 14, 15A, 15B, 15C, 16F, 17F, 18C, 19A, 19F, 20, 21, 22A, 22F, 23A, 23B, 23F, 24F, 27, 28A, 31, 33F, 34, 35A, 35B, 35F, and 38 which are conjugated to a second carrier protein (which is distinct in at least one amino acid from the first carrier protein) together with a pharmaceutically acceptable carrier and an adjuvant.

Formulation of the polysaccharide-protein conjugates of the present invention can be accomplished using art-recognized methods. For instance, 15 individual pneumococcal conjugates can be formulated with a physiologically acceptable vehicle to prepare the composition. Examples of such vehicles include, but are not limited to, water, buffered saline, polyols (e.g., glycerol, propylene glycol, liquid polyethylene glycol) and dextrose solutions.

In a preferred embodiment, the vaccine composition is formulated in L-histidine buffer with sodium chloride.

As defined herein, an “adjuvant” is a substance that serves to enhance the immunogenicity of an immunogenic composition of the invention. An immune adjuvant may enhance an immune response to an antigen that is weakly immunogenic when administered alone, e.g., inducing no or weak antibody titers or cell-mediated immune response, increase antibody titers to the antigen, and/or lowers the dose of the antigen effective to achieve an immune response in the individual. Thus, adjuvants are often given to boost the immune response and are well known to the skilled artisan. Suitable adjuvants to enhance effectiveness of the composition include, but are not limited to:

(1) aluminum salts (alum), such as aluminum hydroxide, aluminum phosphate, aluminum sulfate, etc.;

(2) oil-in-water emulsion formulations (with or without other specific immunostimulating agents such as muramyl peptides (defined below) or bacterial cell wall components), such as, for example, (a) MF59 (International Patent Application Publication No. WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton, Mass.), (b) SAF, containing 10% Squalene, 0.4% Tween 80, 5% pluronic-blocked polymer L121, and thr-MDP either microfluidized into a submicron emulsion or vortexed to generate a larger particle size emulsion, (c) Ribi™ adjuvant system (RAS), (Corixa, Hamilton, Mont.) containing 2% Squalene, 0.2% Tween 80, and one or more bacterial cell wall components from the group consisting of 3-O-deaylated monophosphorylipid A (MPL™) described in U.S. Pat. No. 4,912,094, trehalose dimycolate (TDM), and cell wall skeleton (CWS), preferably MPL+CWS (Detox™); and (d) a Montanide ISA;

(3) saponin adjuvants, such as Quil A or STIMULON™ QS-21 (Antigenics, Framingham, Mass.) (see, e.g., U.S. Pat. No. 5,057,540) may be used or particles generated therefrom such as ISCOM (immunostimulating complexes formed by the combination of cholesterol, saponin, phospholipid, and amphipathic proteins) and Iscomatrix® (having essentially the same structure as an ISCOM but without the protein);

(4) bacterial lipopolysaccharides, synthetic lipid A analogs such as aminoalkyl glucosamine phosphate compounds (AGP), or derivatives or analogs thereof, which are available from Corixa, and which are described in U.S. Pat. No. 6,113,918; one such AGP is 2-[(R)-3-tetradecanoyloxytetradecanoylamino]ethyl 2-Deoxy-4-O-phosphono-3-O—[(R)-3-tetradecanoyloxytetradecanoyl]-2-[(R)-3-tetradecanoyloxytetradecanoylamino]-b-D-glucopyranoside, which is also known as 529 (formerly known as RC529), which is formulated as an aqueous form or as a stable emulsion

(5) synthetic polynucleotides such as oligonucleotides containing CpG motif(s) (U.S. Pat. No. 6,207,646); and

(6) cytokines, such as interleukins (e.g., IL-1, IL-2, IL-4, IL-5, IL-6, IL-7, IL-12, IL-15, IL-18, etc.), interferons (e.g., gamma interferon), granulocyte macrophage colony stimulating factor (GM-CSF), macrophage colony stimulating factor (M-CSF), tumor necrosis factor (TNF), costimulatory molecules B7-1 and B7-2, etc; and

(7) complement, such as a trimer of complement component C3d.

In another embodiment, the adjuvant is a mixture of 2, 3, or more of the above adjuvants, e.g., SBAS2 (an oil-in-water emulsion also containing 3-deacylated monophosphoryl lipid A and QS21).

Muramyl peptides include, but are not limited to, N-acetyl-muramyl-L-threonyl-D-isoglutamine (thr-MDP), N-acetyl-normuramyl-L-alanine-2-(1′-2′ dipalmitoyl-sn-glycero-3-hydroxyphosphoryloxy)-ethylamine (MTP-PE), etc.

In certain embodiments, the adjuvant is an aluminum salt. The aluminum salt adjuvant may be an alum-precipitated vaccine or an alum-adsorbed vaccine. In one embodiment, the vaccine is pre-absorbed on the aluminum adjuvant. Aluminum-salt adjuvants are well known in the art and are described, for example, in Harlow, E. and D. Lane (1988; Antibodies: A Laboratory Manual Cold Spring Harbor Laboratory) and Nicklas, W. (1992; Aluminum salts. Research in Immunology 143:489-493). The aluminum salt includes, but is not limited to, hydrated alumina, alumina hydrate, alumina trihydrate (ATH), aluminum hydrate, aluminum trihydrate, Alhydrogel®, Superfos, Amphogel®, aluminum (III) hydroxide, aluminum hydroxyphosphate sulfate (Aluminum Phosphate Adjuvant (APA)), amorphous alumina, trihydrated alumina, or trihydroxyaluminum.

APA is an aqueous suspension of aluminum hydroxyphosphate. APA is manufactured by blending aluminum chloride and sodium phosphate in a 1:1 volumetric ratio to precipitate aluminum hydroxyphosphate. After the blending process, the material is size-reduced with a high-shear mixer to achieve a monodisperse particle size distribution. The product is then diafiltered against physiological saline and steam sterilized.

In certain embodiments, a commercially available Al(OH)₃ (e.g. Alhydrogel® or Superfos of Denmark/Accurate Chemical and Scientific Co., Westbury, N.Y.) is used to adsorb proteins in a ratio of 50-1000 μg protein/mg aluminum hydroxide. Adsorption of protein is dependent, in another embodiment, on the pI (Isoelectric pH) of the protein and the pH of the medium. A protein with a lower pI adsorbs to the positively charged aluminum ion more strongly than a protein with a higher pI. Aluminum salts may establish a depot of Ag that is released slowly over a period of 2-3 weeks, be involved in nonspecific activation of macrophages and complement activation, and/or stimulate innate immune mechanism (possibly through stimulation of uric acid). See, e.g., Lambrecht et al., 2009, Curr Opin Immunol 21:23.

Monovalent bulk aqueous conjugates are typically blended together and diluted. Once diluted, the batch is sterile filtered. Aluminum phosphate adjuvant is added aseptically to target a final concentration of 4 μg/mL for all serotypes except 6B, which is diluted to target 8 g/mL, and a final aluminum concentration of 250 μg/mL. The adjuvanted, formulated batch will be filled into vials or syringes.

In certain embodiments, the adjuvant is a CpG-containing nucleotide sequence, for example, a CpG-containing oligonucleotide, in particular, a CpG-containing oligodeoxynucleotide (CpG ODN). In another embodiment, the adjuvant is ODN 1826, which may be acquired from Coley Pharmaceutical Group.

“CpG-containing nucleotide,” “CpG-containing oligonucleotide,” “CpG oligonucleotide,” and similar terms refer to a nucleotide molecule of 6-50 nucleotides in length that contains an unmethylated CpG moiety. See, e.g., Wang et al., 2003, Vaccine 21:4297. In another embodiment, any other art-accepted definition of the terms is intended. CpG-containing oligonucleotides include modified oligonucleotides using any synthetic internucleoside linkages, modified base and/or modified sugar.

Methods for use of CpG oligonucleotides are well known in the art and are described, for example, in Sur et al., 1999, J Immunol. 162:6284-93; Verthelyi, 2006, Methods Mol Med. 127:139-58; and Yasuda et al., 2006, Crit Rev Ther Drug Carrier Syst. 23:89-110.

### Administration/Dosage

The compositions and formulations of the present invention can be used to protect or treat a human susceptible to infection, e.g., a pneumococcal infection, by means of administering the vaccine via a systemic or mucosal route. In one embodiment, the present invention provides a method of inducing an immune response to a S. pneumoniae capsular polysaccharide conjugate, comprising administering to a human an immunologically effective amount of an immunogenic composition of the present invention. In another embodiment, the present invention provides a method of vaccinating a human against a pneumococcal infection, comprising the step of administering to the human an immunogically effective amount of an immunogenic composition of the present invention.

Optimal amounts of components for a particular vaccine can be ascertained by standard studies involving observation of appropriate immune responses in subjects. For example, in another embodiment, the dosage for human vaccination is determined by extrapolation from animal studies to human data. In another embodiment, the dosage is determined empirically. Infant Rhesus Monkey animal data provided in the Examples demonstrates that that the vaccine is immunogenic.

“Effective amount” of a composition of the invention refers to a dose required to elicit antibodies that significantly reduce the likelihood or severity of infectivity of a microbe, e.g., S. pneumoniae, during a subsequent challenge.

The methods of the invention can be used for the prevention and/or reduction of primary clinical syndromes caused by microbes, e.g., S. pneumoniae, including both invasive infections (meningitis, pneumonia, and bacteremia), and noninvasive infections (acute otitis media, and sinusitis).

Administration of the compositions of the invention can include one or more of: injection via the intramuscular, intraperitoneal, intradermal or subcutaneous routes; or via mucosal administration to the oral/alimentary, respiratory or genitourinary tracts. In one embodiment, intranasal administration is used for the treatment of pneumonia or otitis media (as nasopharyngeal carriage of pneumococci can be more effectively prevented, thus attenuating infection at its earliest stage).

The amount of conjugate in each vaccine dose is selected as an amount that induces an immunoprotective response without significant, adverse effects. Such amount can vary depending upon the pneumococcal serotype. Generally, for polysaccharide-based conjugates, each dose will comprise 0.1 to 100 g of each polysaccharide, particularly 0.1 to g, and more particularly 1 to 5 μg. For example, each dose can comprise 100, 150, 200, 250, 300, 400, 500, or 750 ng or 1, 1.5, 2, 3, 4, 5, 6, 7, 7.5, 8, 9, 10, 11, 12, 13, 14, 15, 16, 18, 20, 22, 25, 30, 40, 50, 60, 70, 80, 90, or 100 g.

Optimal amounts of components for a particular vaccine can be ascertained by standard studies involving observation of appropriate immune responses in subjects. For example, in another embodiment, the dosage for human vaccination is determined by extrapolation from animal studies to human data. In another embodiment, the dosage is determined empirically.

In one embodiment, the dose of the aluminum salt is 10, 15, 20, 25, 30, 50, 70, 100, 125, 150, 200, 300, 500, or 700 μg, or 1, 1.2, 1.5, 2, 3, 5 mg or more. In yet another embodiment, the dose of aluminum salt described above is per g of recombinant protein.

In a particular embodiment of the present invention, the PCV15 vaccine is a sterile formulation of pneumococcal capsular polysaccharides of serotypes 1, 3, 4, 5, 6A, 6B, 7F, 9V, 14, 18C, 19A, 19F, 22F, 23F and 33F individually conjugated to CRM₁₉₇. In one aspect, each dose is formulated to contain: 4 μg/mL or 8 μg/mL of each saccharide, except for 6B at 8 μg/mL or 16 μg/mL; and about 64 μg/mL or 128 μg/mL CRM₁₉₇ carrier protein. In one aspect, each 0.5 mL dose is formulated to contain: 2 g of each saccharide, except for 6B at 4 μg; about 32 μg CRM₁₉₇ carrier protein (e.g., 32 μg±5 μg, ±3 μg, ±2 μg, or ±1 μg), 0.125 mg of elemental aluminum (0.5 mg aluminum phosphate) adjuvant; and sodium chloride and L-histidine buffer. The sodium chloride concentration is about 150 mM (e.g., 150 mM±25 mM, ±20 mM, ±15 mM, ±10 mM, or ±5 mM) and about 20 mM (e.g, 20 mM±5 mM, ±2.5 mM, ±2 mM, ±1 mM, or ±0.5 mM) L-histidine buffer.

According to any of the methods of the present invention and in one embodiment, the subject is human. In certain embodiments, the human subject is an infant (less than 1 year of age), toddler (approximately 12 to 24 months), or young child (approximately 2 to 5 years). In other embodiments, the human subject is an elderly patient (>65 years). The compositions of this invention are also suitable for use with older children, adolescents and adults (e.g., aged 18 to 45 years or 18 to 65 years).

In one embodiment of the methods of the present invention, a composition of the present invention is administered as a single inoculation. In another embodiment, the vaccine is administered twice, three times or four times or more, adequately spaced apart. For example, the composition may be administered at 1, 2, 3, 4, 5, or 6 month intervals or any combination thereof. The immunization schedule can follow that designated for pneumococcal vaccines. For example, the routine schedule for infants and toddlers against invasive disease caused by S. pneumoniae is 2, 4, 6 and 12-15 months of age. Thus, in a preferred embodiment, the composition is administered as a 4-dose series at 2, 4, 6, and 12-15 months of age.

The compositions of this invention may also include one or more proteins from S. pneumoniae. Examples of S. pneumoniae proteins suitable for inclusion include those identified in International Patent Application Publication Nos. WO 02/083855 and WO 02/053761.

### Formulations

The compositions of the invention can be administered to a subject by one or more method known to a person skilled in the art, such as parenterally, transmucosally, transdermally, intramuscularly, intravenously, intra-dermally, intra-nasally, subcutaneously, intra-peritonealy, and formulated accordingly.

In one embodiment, compositions of the present invention are administered via epidermal injection, intramuscular injection, intravenous, intra-arterial, subcutaneous injection, or intra-respiratory mucosal injection of a liquid preparation. Liquid formulations for injection include solutions and the like. The composition of the invention can be formulated as single dose vials, multi-dose vials or as pre-filled syringes.

In another embodiment, compositions of the present invention are administered orally, and are thus formulated in a form suitable for oral administration, i.e., as a solid or a liquid preparation. Solid oral formulations include tablets, capsules, pills, granules, pellets and the like. Liquid oral formulations include solutions, suspensions, dispersions, emulsions, oils and the like.

In one aspect of the invention, the formulation is a solid dried formulation prepared from lyophilization, freezing, microwave drying or through the generation of lyospheres. The formulations can be stored at −70° C., −20° C., 2-8° C. or at room temperature. The dried formulations can be expressed in terms of the weight of the components in a unit dose vial, but this varies for different doses or vial sizes. Alternatively, the dried formulations of the present invention can be expressed in the amount of a component as the ratio of the weight of the component compared to the weight of the drug substance (DS) in the same sample (e.g. a vial). This ratio may be expressed as a percentage. Such ratios reflect an intrinsic property of the dried formulations of the present invention, independent of vial size, dosing, and reconstitution protocol. In one embodiment, the formulation has a d(0.5) m less than 20, 15, 10 or 5 m. In other embodiments, the formulation is in lyospheres.

In another aspect of the invention, the formulation is a reconstituted solution. A dried solid formulation can be reconstituted at different concentrations depending on clinical factors, such as route of administration or dosing. For example, a dried formulation may be reconstituted at a high concentration (i.e. in a small volume) if necessary for subcutaneous administration. High concentrations may also be necessary if high dosing is required for a particular subject, particularly if administered subcutaneously where injection volume must be minimized. Subsequent dilution with water or isotonic buffer can then readily be used to dilute the drug product to a lower concentration. If isotonicity is desired at lower drug product concentration, the dried powder may be reconstituted in the standard low volume of water and then further diluted with isotonic diluent, such as 0.9% sodium chloride.

Reconstitution generally takes place at a temperature of about 25° C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g. phosphate-buffered saline), sterile saline solution, Ringer's solution or dextrose solution. The reconstitution volume can be about 0.5-1.0 ml, preferably 0.5 ml or 0.7 ml.

In another embodiment of the invention, the formulation is the aqueous solution prepared before lyophilization, freezing, microwave drying or generation of lyospheres.

The pharmaceutical composition may be isotonic, hypotonic or hypertonic. However it is often preferred that a pharmaceutical composition for infusion or injection is essentially isotonic, when it is administered. Hence, for storage the pharmaceutical composition may preferably be isotonic or hypertonic. If the pharmaceutical composition is hypertonic for storage, it may be diluted to become an isotonic solution prior to administration.

The isotonic agent may be an ionic isotonic agent such as a salt or a non-ionic isotonic agent such as a carbohydrate. Examples of ionic isotonic agents include but are not limited to NaCl, CaCl₂, KCl and MgCl₂.

It is also preferred that at least one pharmaceutically acceptable additive is a buffer. For some purposes, for example, when the pharmaceutical composition is meant for infusion or injection, it is often desirable that the composition comprises a buffer, which is capable of buffering a solution to a pH in the range of 4 to 10, such as 5 to 9, for example 6 to 8.

The buffer may for example be selected from the group consisting of Tris, acetate, glutamate, lactate, maleate, tartrate, phosphate, citrate, carbonate, glycinate, L-histidine, glycine, succinate and triethanolamine buffer.

The buffer may furthermore for example be selected from USP compatible buffers for parenteral use, in particular, when the pharmaceutical formulation is for parenteral use. For example the buffer may be selected from the group consisting of monobasic acids such as acetic, benzoic, gluconic, glyceric and lactic; dibasic acids such as aconitic, adipic, ascorbic, carbonic, glutamic, malic, succinic and tartaric, polybasic acids such as citric and phosphoric; and bases such as ammonia, diethanolamine, glycine, triethanolamine, and Tris.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, glycols such as propylene glycols or polyethylene glycol, Polysorbate 80 (PS-80), Polysorbate 20 (PS-20), and Poloxamer 188 (P188) are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

The formulations of the invention may also contain a surfactant. Preferred surfactants include, but are not limited to: the polyoxyethylene sorbitan esters surfactants (commonly referred to as the Tweens), especially PS-20 and PS-80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. A preferred surfactant for including in the emulsion is PS-80.

Mixtures of surfactants can be used, e.g. PS-80/Span 85 mixtures. A combination of a polyoxyethylene sorbitan ester such as polyoxyethylene sorbitan monooleate (PS-80) and an octoxynol such as t-octylphenoxypolyethoxyethanol (Triton X-100) is also suitable. Another useful combination comprises laureth 9 plus a polyoxyethylene sorbitan ester and/or an octoxynol.

Preferred amounts of surfactants are: polyoxyethylene sorbitan esters (such as PS-80) 0.01 to 1% w/v, in particular about 0.1% w/v; octyl- or nonylphenoxy polyoxyethanols (such as Triton X-100, or other detergents in the Triton series) 0.001 to 0.1% w/v, in particular 0.005 to 0.02% w/v; polyoxyethylene ethers (such as laureth 9) 0.1 to 20% w/v, preferably 0.1 to 10% w/v and in particular 0.1 to 1% w/v or about 0.5% w/v.

In certain embodiments, the surfactant is polysorbate 20 (IUPAC name: Polyoxyethylene (20) sorbitan monolaurate; PS-20) is a commercially available surfactant, commonly referred to as the Tween® 20. In certain embodiments, the final concentration of the polysorbate 20 in the formulations of the invention is in the range from 0.001% to 10% w/v, from 0.025% to 2.5% w/v, from 0.1% to 0.2% w/v or 0.025% to 0.1% w/v.

In certain embodiments, the surfactant is a poloxamer having a molecular weight in the range from 1100 Da to 17,400 Da.

A poloxamer is a nonionic triblock copolymer composed of a central hydrophobic chain of polyoxypropylene (poly(propylene oxide)) flanked by two hydrophilic chains of polyoxyethylene (poly(ethylene oxide)). Poloxamers are also known by the tradename Pluronic*. Because the lengths of the polymer blocks can be customized, many different poloxamers exist that have slightly different properties. For the generic term “poloxamer”, these copolymers are commonly named with the letter “P” (for poloxamer) followed by three digits, the first two digits×100 give the approximate molecular mass of the polyoxypropylene core, and the last digit×10 gives the percentage polyoxyethylene content (e.g., P407=Poloxamer with a polyoxypropylene molecular mass of 4,000 g/mol and a 70% polyoxyethylene content). For the Pluronic® tradename, coding of these copolymers starts with a letter to define its physical form at room temperature (L=liquid, P=paste, F=flake (solid)) followed by two or three digits. The first digit (two digits in a three-digit number) in the numerical designation, multiplied by 300, indicates the approximate molecular weight of the hydrophobe; and the last digit×10 gives the percentage polyoxyethylene content (e.g., L61=Pluronic® with a polyoxypropylene molecular mass of 1,800 g/mol and a 10% polyoxyethylene content). See U.S. Pat. No. 3,740,421.

Examples of poloxamers have the general formula:

HO(C₂H₄O)ₐ(C₃H₆O)(C₂H₄O)ₐH,

wherein a and b blocks have the following values:

| Pluronic ® | Poloxamer | a | b | Molecular Weight |
|---|---|---|---|---|
| L31 | | 2 | 16 | 1100 (average) |
| L35 | | | | 1900 (average) |
| L44NF | 124 | 12 | 20 | 2090 to 2360 |
| L64 | | | | 2900 (average) |
| L81 | | | | 2800 (average) |
| L121 | | | | 4400 (average) |
| P123 | | 20 | 70 | 5750 (average) |
| F68NF | 188 | 80 | 27 | 7680 to 9510 |
| F87NF | 237 | 64 | 37 | 6840 to 8830 |
| F108NF | 338 | 141 | 44 | 12700 to 17400 |
| F127NF | 407 | 101 | 56 | 9840 to 14600 |

Molecular weight units, as used herein, are in Dalton (Da) or g/mol.

For the formulations, a poloxamer generally has a molecular weight in the range from 1100 Da to 17,400 Da, from 7,500 Da to 15,000 Da, or from 7,500 Da to 10,000 Da. The poloxamer can be selected from poloxamer 188 or poloxamer 407. The final concentration of the poloxamer in the formulations of the invention is from 0.001 to 5% w/v, or 0.025 to 1% w/v.

Suitable polymers for the formulations are polymeric polyols, particularly polyether diols including, but are not limited to, propylene glycol and polyethylene glycol, Polyethylene glycol monomethyl ethers. Propylene glycol is available in a range of molecular weights of the monomer from ˜425 to ˜2700. Polyethylene glycol and Polyethylene glycol monomethyl ether is also available in a range of molecular weights ranging from ˜200 to ˜35000 including but not limited to PEG200, PEG300, PEG400, PEG1000 PEG MME 550, PEG MME 600, PEG MME 2000, PEG MME 3350 and PEG MME 4000. A preferred polyethylene glycol is polyethylene glycol 400. The final concentration of the polymer in the formulations of the invention may be 1 to 20% w/v or 6 to 20% w/v.

The formulation also contains a pH-buffered saline solution. The buffer may, for example, be selected from the group consisting of Tris, acetate, glutamate, lactate, maleate, tartrate, phosphate, citrate, carbonate, glycinate, L-histidine, glycine, succinate, HEPES (4-(2-hydroxyethyl)-1-piperazineethanesulfonic acid), MOPS (3-(N-morpholino)propanesulfonic acid), MES (2-(N-morpholino)ethanesulfonic acid) and triethanolamine buffer. The buffer is capable of buffering a solution to a pH in the range of 4 to 10, 5.2 to 7.5, or 5.8 to 7.0. In certain aspect of the invention, the buffer selected from the group consisting of phosphate, succinate, L-histidine, MES, MOPS, HEPES, acetate or citrate. The buffer may furthermore, for example, be selected from USP compatible buffers for parenteral use, in particular, when the pharmaceutical formulation is for parenteral use. The concentrations of buffer will range from 1 mM to 50 mM or 5 mM to 50 mM. In certain aspects, the buffer is L-histidine at a final concentration of 5 mM to 50 mM, or succinate at a final concentration of 1 mM to 10 mM. In certain aspects, the L-histidine is at a final concentration of 20 mM±2 mM.

While the saline solution (i.e., a solution containing NaCl) is preferred, other salts suitable for formulation include but are not limited to, CaCl₂, KCl and MgCl₂ and combinations thereof. Suitable salt ranges include, but not are limited to 25 mM to 500 mM or 40 mM to 170 mM. In one aspect, the saline is NaCl, optionally present at a concentration from 20 mM to 170 mM. In a preferred embodiment, the formulations comprise a L-histidine buffer with sodium chloride.

In certain embodiments of the formulations described herein, the polysaccharide-protein conjugates comprise one or more pneumococcal polysaccharides conjugated to a carrier protein. The carrier protein can be selected from CRM₁₉₇, diphtheria toxin fragment B (DTFB), DTFBC8, Diphtheria toxoid (DT), tetanus toxoid (TT), fragment C of TT, pertussis toxoid, cholera toxoid, E. coli LT, E. coli ST, exotoxin A from Pseudomonas aeruginosa, and combinations thereof. In certain aspects, one or more of the polysaccharide-protein conjugates are conjugated to DTFB. In one aspect, all of the polysaccharide-protein conjugates are prepared using aqueous chemistry. As an example, the polysaccharide-protein conjugate formulation can be a 15-valent pneumococcal conjugate (15vPnC) formulation consisting essentially of S. pneumoniae polysaccharide from serotypes 1, 3, 4, 5, 6A, 6B, 7F, 9V, 14, 18C, 19A, 19F, 22F, 23F and 33F conjugated to a CRM₁₉₇ polypeptide. In another aspect, one or more of the polysaccharide protein conjugates is prepared using DMSO chemistry. As an example, the polysaccharide-protein conjugate formulation can be a 15-valent pneumococcal conjugate (15vPnC) formulation wherein polysaccharide protein conjugates from serotypes 6A, 6B, 7F, 18C, 19A, 19F, and 23F are prepared using DMSO chemistry and polysaccharide protein conjugates from serotypes 1, 3, 4, 5, 9V, 14, 22F, and 33F are prepared using aqueous chemistry.

In another embodiment, the pharmaceutical composition is delivered in a controlled release system. For example, the agent can be administered using intravenous infusion, a transdermal patch, liposomes, or other modes of administration. In another embodiment, polymeric materials are used; e.g. in microspheres in or an implant.

The compositions of this invention may also include one or more proteins from S. pneumoniae. Examples of S. pneumoniae proteins suitable for inclusion include those identified in International Patent Application Publication Nos. WO 02/083855 and WO 02/053761.

### Processes for Preparing the Lyospheres

In some embodiments, the unitary volumes containing the aqueous medium mixture are formed on a solid element containing cavities. The solid element is cooled below the freezing temperature of the mixture, the cavities are filled with the mixture, and the mixture is solidified while present in the cavity to form the unitary forms. The unitary forms are dried in a vacuum to provide the lyospheres. U.S. Pat. No. 9,119,794, the disclosure of which is herein incorporated by reference, discloses similar processes for forming lyospheres.

In other embodiments, the lyospheres are formed in a substantially spherical shape and are prepared by freezing droplets of a liquid composition of a desired biological material on a flat, solid surface, in particular, a surface that does not have any cavities, followed by lyophilizing the unitary forms. U.S. Patent Application Publication No. US2014/0294872, the disclosure of which is herein incorporated by reference, discloses similar processes for forming lyospheres.

Briefly, in some embodiments the process comprises dispensing at least one liquid droplet having a substantially spherical shape onto a solid and flat surface (i.e., lacking any sample wells or cavity), freezing the droplet on the surface without contacting the droplet with a cryogenic substance and lyophilizing the frozen droplet to produce a dried pellet that is substantially spherical in shape. The process may be used in a high throughput mode to prepare multiple dried pellets by simultaneously dispensing the desired number of droplets onto the solid, flat surface, freezing the droplets and lyophilizing the frozen droplets. Pellets prepared by this process from a liquid formulation may have a high concentration of a biological material (such as a protein therapeutic) and may be combined into a set of dried pellets.

In some embodiments, the solid, flat surface is the top surface of a metal plate which comprises a bottom surface that is in physical contact with a heat sink adapted to maintain the top surface of the metal plate at a temperature of −90° C. or below. Since the top surface of the metal plate is well below the freezing point of the liquid formulation, the droplet freezes essentially instantaneously with the bottom surface of the droplet touching the top surface of the metal plate.

In other embodiments, the solid, flat surface is hydrophobic and comprises the top surface of a thin film that is maintained above 0° C. during the dispensing step. The dispensed droplet is frozen by cooling the thin film to a temperature below the freezing temperature of the formulation.

### Lyophilization Process

The lyophilized formulations of the present invention are formed by lyophilization (freeze-drying) of a pre-lyophilization solution. Freeze-drying is accomplished by freezing the formulation and subsequently subliming water at a temperature suitable for primary drying. Under this condition, the product temperature is below the eutectic point or the collapse temperature of the formulation. Typically, the shelf temperature for the primary drying will range from about −50 to 25° C. (provided the product remains frozen during primary drying) at a suitable pressure, ranging typically from about 30 to 250 mTorr. The formulation, size and type of the container holding the sample (e.g., glass vial) and the volume of liquid will dictate the time required for drying, which can range from a few hours to several days (e.g. 40-60 hrs). A secondary drying stage may be carried out at about 0-40° C., depending primarily on the type and size of container and the type of protein employed. The secondary drying time is dictated by the desired residual moisture level in the product and typically takes at least about 5 hours. Typically, the moisture content of a lyophilized formulation is less than about 5%, and preferably less than about 3%. The pressure may be the same as that employed during the primary drying step. Freeze-drying conditions can be varied depending on the formulation, vial size and lyophilization trays.

In some instances, it may be desirable to lyophilize or microwave dry the protein-polysaccharide formulation in the container in which reconstitution is to be carried out in order to avoid a transfer step. The container in this instance may, for example, be a 2, 3, 5, 10 or 20 ml vial.

Having described various embodiments of the invention with reference to the accompanying description and drawings, it is to be understood that the invention is not limited to those precise embodiments, and that various changes and modifications may be effected therein by one skilled in the art without departing from the scope or spirit of the invention as defined in the appended claims.

The following examples illustrate, but do not limit the invention.

### EXAMPLES

### Example 1: Preparation of S. pneumoniae Capsular Polysaccharides

Methods of culturing pneumococci are well known in the art. See, e.g., Chase, 1967, Methods of Immunology and Immunochemistry 1:52. Methods of preparing pneumococcal capsular polysaccharides are also well known in the art. See, e.g., European Patent No. EP0497524. Isolates of pneumococcal subtypes are available from the American Type Culture Collection (Manassas, Va.). The bacteria are identified as encapsulated, non-motile, Gram-positive, lancet-shaped diplococci that are alpha-hemolytic on blood-agar. Subtypes can be differentiated on the basis of Quelling reaction using specific antisera. See, e.g., U.S. Pat. No. 5,847,112.

Cell banks representing each of the S. pneumoniae serotypes of interest were obtained from the Merck Culture Collection (Rahway, N.J.) in a frozen vial. A thawed seed culture was transferred to the seed fermentor containing a pre-sterilized growth media appropriate for S. pneumoniae. The culture was grown in the seed fermentor with temperature and pH control. The entire volume of the seed fermentor was transferred to a production fermentor containing pre-sterilized growth media. The production fermentation was the final cell growth stage of the process. Temperature, pH, and the agitation rate were controlled.

The fermentation process was terminated via the addition of an inactivating agent. After inactivation, the batch was transferred to the inactivation tank where it was held at controlled temperature and agitation. Cell debris was removed using a combination of centrifugation and filtration. The batch was ultrafiltered and diafiltered. The batch was then subjected to solvent-based fractionations that remove impurities and recover polysaccharide.

### Example 2: Conjugation of Serotypes 1, 3, 4, 5, 6A, 6B, 7F, 9V, 14, 18C, 19A, 19F, 22F, 23F, and 33F to CRM₁₉₇ Using Reductive Amination in Aqueous Solution

The different serotype polysaccharides were individually conjugated to purified CRM₁₉₇ carrier protein using a common process flow. Polysaccharide was dissolved, size reduced, chemically activated and buffer-exchanged by ultrafiltration. Purified CRM₁₉₇ was then conjugated to the activated polysaccharide utilizing NiCl₂ (2 mM) in the reaction mixture, and the resulting conjugate was purified by ultrafiltration prior to a final 0.2 micron filtration. Several process parameters within each step, such as pH, temperature, concentration, and time were controlled to serotype-specific values in section below.

### Polysaccharide Size Reduction and Oxidation

Purified pneumococcal capsular polysaccharide powder was dissolved in water, and all serotypes, except serotype 19A, were 0.45-micron filtered. Serotypes 1, 3, 4, 5, 6A, 6B, 7F, 9V, 14, 19A, 19F, 22F, 23F, and 33F were homogenized to reduce the molecular mass of the polysaccharide. Serotype 18C was size-reduced by either homogenization or acid hydrolysis at ≥90° C. Serotype 19A was not size reduced due to its relatively low starting size. Homogenization pressure and number of passes through the homogenizer were controlled to serotype-specific targets (150-1000 bar; 4-7 passes) to achieve a serotype-specific molecular mass. Size-reduced polysaccharide was 0.2-micron filtered and then concentrated and diafiltered against water using a 10 kDa NMWCO tangential flow ultrafiltration membrane. A 5 kDa NMWCO membrane was used for acid-hydrolyzed serotype 18C.

The polysaccharide solution was then adjusted to a serotype-specific temperature (4-22° C.) and pH (4-5) with a sodium acetate buffer to minimize polysaccharide size reduction due to activation. For all serotypes (except serotype 4), polysaccharide activation was initiated with the addition of a 100 mM sodium metaperiodate solution. The amount of sodium metaperiodate added was serotype-specific, ranging from approximately 0.1 to 0.5 moles of sodium metaperiodate per mole of polysaccharide repeating unit. The serotype-specific charge of sodium metaperiodate was to achieve a target level of polysaccharide activation (moles aldehyde per mole of polysaccharide repeating unit). For serotype 4, prior to the sodium metaperiodate addition, the batch was incubated at approximately 50° C. and pH 4.1 to partially deketalize the polysaccharide.

For all serotypes, with the exception of serotypes 5 and 7F, the activated product was diafiltered against 10 mM potassium phosphate, pH 6.4 using a 10 kDa NMWCO tangential flow ultrafiltration membrane. A 5 kDa NMWCO membrane was used for acid-hydrolyzed serotype 18C. Serotypes 5 and 7F were diafiltered against 10 mM sodium acetate. Ultrafiltration for all serotypes was conducted at 2-8° C.

### Polysaccharide Conjugation to CRM₁₉₇

Oxidized polysaccharide solution was mixed with water and 1.5 M potassium phosphate, pH 6.0 or pH 7.0, depending on the serotype. The buffer pH selected was to improve the stability of activated polysaccharide during the conjugation reaction. Purified CRM₁₉₇, obtained through expression in Pseudomonas fluorescens as previously described (See International Patent Application Publication No. WO 2012/173876 A1), was 0.2-micron filtered and combined with the buffered polysaccharide solution at a polysaccharide to CRM₁₉₇ mass ratio ranging from 0.4 to 1.0 w/v depending on the serotype. The mass ratio was selected to control the polysaccharide to CRM₁₉₇ ratio in the resulting conjugate. The polysaccharide and phosphate concentrations were serotype-specific, ranging from 3.6 to 10.0 g/L and 100 to 150 mM, respectively, depending on the serotype. The serotype-specific polysaccharide concentration was selected to control the size of the resulting conjugate. The solution was then 0.2-micron filtered. Nickel chloride was added to approximately 2 mM using a 100 mM nickel chloride solution. Sodium cyanoborohydride (2 moles per mole of polysaccharide repeating unit) was added. Conjugation proceeded for a serotype-specific duration (72 to 120 hours) to maximize consumption of polysaccharide and protein.

Acid-hydrolyzed serotype 18C was conjugated at 37° C. in 100 mM potassium phosphate at approximately pH 8 with sodium cyanoborohydride using polysaccharide and protein concentrations of approximately 12.0 g/L and 6.0 g/L, respectively.

Reduction with Sodium Borohydride

Following the conjugation reaction, the batch was diluted to a polysaccharide concentration of approximately 3.5 g/L, cooled to 2-8° C., and 1.2-micron filtered. All serotypes (except serotype 5) were diafiltered against 100 mM potassium phosphate, pH 7.0 at 2-8° C. using a 100 kDa NMWCO tangential flow ultrafiltration membrane. The batch, recovered in the retentate, was then diluted to approximately 2.0 g polysaccharide/L and pH-adjusted with the addition of 1.2 M sodium bicarbonate, pH 9.4. Sodium borohydride (1 mole per mole of polysaccharide repeating unit) was added. 1.5 M potassium phosphate, pH 6.0 was later added. Serotype 5 was diafiltered against 300 mM potassium phosphate using a 100 kDa NMWCO tangential flow ultrafiltration membrane.

### Final Filtration and Product Storage

The batch was then concentrated and diafiltered against 10 mM L-histidine in 150 mM sodium chloride, pH 7.0 at 4° C. using a 300 kDa NMWCO tangential flow ultrafiltration membrane. The retentate batch was 0.2 micron filtered.

Serotype 19F was incubated for approximately 7 days at 22° C., diafiltered against 10 mM L-histidine in 150 mM sodium chloride, pH 7.0 at 4° C. using a 100 kDa NMWCO tangential flow ultrafiltration membrane, and 0.2-micron filtered.

The batch was adjusted to a polysaccharide concentration of 1.0 g/L with additional 10 mM L-histidine in 150 mM sodium chloride, pH 7.0. The batch was dispensed into aliquots and frozen at ≤−60° C.

### Example 3: Methods for the Conjugation of Serotypes 6A, 6B, 7F, 18C, 19A, 19F, and 23F to CRM₁₉₇ Using Reductive Amination in Dimethylsulfoxide

The different serotype polysaccharides were individually conjugated to purified CRM₁₉₇ carrier protein using a common process flow. Polysaccharide was dissolved, sized to a target molecular mass, chemically activated and buffer-exchanged by ultrafiltration. Activated polysaccharide and purified CRM₁₉₇ were individually lyophilized and redissolved in dimethylsulfoxide (DMSO). Redissolved polysaccharide and CRM₁₉₇ solutions were then combined and conjugated as described below. The resulting conjugate was purified by ultrafiltration prior to a final 0.2-micron filtration. Several process parameters within each step, such as pH, temperature, concentration, and time were controlled to serotype-specific values in section below.

### Polysaccharide Size Reduction and Oxidation

Purified pneumococcal capsular polysaccharide powder was dissolved in water, and all serotypes, except serotype 19A, were 0.45-micron filtered. All serotypes, except serotypes 18C and 19A, were homogenized to reduce the molecular mass of the polysaccharide. Homogenization pressure and number of passes through the homogenizer were controlled to serotype-specific targets (150-1000 bar; 4-7 passes). Serotype 18C was size-reduced by acid hydrolysis at ≥90° C. Serotype 19A was not sized-reduced.

Size-reduced polysaccharide was 0.2-micron filtered and then concentrated and diafiltered against water using a 10 kDa NMWCO tangential flow ultrafiltration membrane. A 5 kDa NMWCO membrane was used for serotype 18C.

The polysaccharide solution was then adjusted to a serotype-specific temperature (4-22° C.) and pH (4-5) with a sodium acetate buffer. Polysaccharide activation was initiated with the addition of a sodium metaperiodate solution. The amount of sodium metaperiodate added was serotype-specific, ranging from approximately 0.1 to 0.5 moles of sodium metaperiodate per mole of polysaccharide repeating unit.

For all serotypes, the activated product was diafiltered against 10 mM potassium phosphate, pH 6.4 using a 10 kDa NMWCO tangential flow ultrafiltration membrane. A 5 kDa NMWCO membrane was used for serotype 18C. Ultrafiltration for all serotypes was conducted at 2-8° C.

### Polysaccharide Conjugation to CRM₁₉₇

Purified CRM₁₉₇, obtained through expression in Pseudomonas fluorescens as previously described (See International Patent Application Publication No. WO 2012/173876 A1), was diafiltered against 2 mM phosphate, pH 7 buffer using a 5 kDa NMWCO tangential flow ultrafiltration membrane and 0.2-micron filtered.

The oxidized polysaccharide solution was formulated with water and sucrose in preparation for lyophilization. The protein solution was formulated with water, phosphate buffer, and sucrose in preparation for lyophilization to achieve optimal redissolution in DMSO following lyophilization.

Formulated polysaccharide and CRM₁₉₇ solutions were individually lyophilized. Lyophilized polysaccharide and CRM₁₉₇ materials were redissolved in DMSO and combined using a tee mixer. Sodium cyanoborohydride (1 mole per mole of polysaccharide repeating unit) was added, and conjugation proceeded for a serotype-specific duration (1 to 48 hours) to achieve a targeted conjugate size.

Reduction with Sodium Borohydride

Sodium borohydride (2 mole per mole of polysaccharide repeating unit) was added following the conjugation reaction. The batch was diluted into 150 mM sodium chloride at approximately 4° C. Potassium phosphate buffer was then added to neutralize the pH. The batch was concentrated and diafiltered at approximately 4° C. against 150 mM sodium chloride using a 10 kDa NMWCO tangential flow ultrafiltration membrane.

### Final Filtration and Product Storage

Each batch was then concentrated and diafiltered against 10 mM L-histidine in 150 mM sodium chloride, pH 7.0 at 4° C. using a 300 kDa NMWCO tangential flow ultrafiltration membrane. The retentate batch was 0.2-micron filtered.

Serotype 19F was incubated for approximately 5 days, diafiltered against 10 mM L-histidine in 150 mM sodium chloride, pH 7.0 at approximately 4° C. using a 300 kDa NMWCO tangential flow ultrafiltration membrane, and 0.2-micron filtered.

The batch was diluted with additional 10 mM L-histidine in 150 mM sodium chloride, pH 7.0 and dispensed into aliquots and frozen at ≤−60° C.

### Example 4: Formulations of a 15-Valent Pneumococcal Conjugate Vaccine

Pneumococcal polysaccharide-protein conjugates prepared as described above were used for the formulation of a 15-valent pneumococcal conjugate vaccine (PCV15) having serotypes 1, 3, 4, 5, 6A, 6B, 7F, 9V, 14, 18C, 19A, 19F, 22F, 23F, and 33F. The formulations were prepared using pneumococcal polysaccharide-CRM₁₉₇ conjugates generated by reductive amination in aqueous solutions (Example 3).

### Formulation Excipient Stock Preparation

Thirteen concentrated excipient stocks were prepared with a combination of excipients, resulting in the final vaccine drug product formulation concentrations as listed in Table 1; in a final base formulation of 20 mM Histidine, 150 mM NaCl, 0.2% w/v PS20 (2 mg/ml), pH 5.8.

Histidine, PEG400, Hydroxypropylmethyl cellulose (HPMC), 2-hydroxyethyl cellulose (2-HEC), and Hydroxypropyl cellulose (HPC) were purchased from Sigma-Aldrich, St. Louis Mo. Carboxymethyl cellulose (CMC) and PEO100K were purchased from Acros Organics. Sodium Chloride, Polysorbate 20, Mannitol, Sucrose, Propylene glycol, and Glycerol were purchased from Fisher Scientific.

TABLE 1
PCV Formulation Compositions

| Key | Excipients | Concentration | | mg/vial |
|---|---|---|---|---|
| Fl | Mannitol | 50 | mg/mL | 3.5 |
| | Sucrose | 20 | mg/mL | 14 |
| | Aluminum Phosphate | 0.25 | mg/mL | 0.18 |
| | Adjuvant (APA) | | | |
| | Polysorbate 20 | 2 | mg/mL | 1.4 |
| | Sodium Chloride | 150 | mM | 6.3 |
| | L-Histidine | 20 | mM | 2.2 |
| F2 | Mannitol | 60 | mg/mL | 42 |
| | Sucrose | 40 | mg/mL | 28 |
| | Aluminum Phosphate | 0.25 | mg/mL | 0.18 |
| | Adjuvant (APA) | | | |
| | Polysorbate 20 | 2 | mg/mL | 1.4 |
| | Sodium Chloride | 150 | mM | 6.3 |
| | L-Histidine | 20 | mM | 2.2 |
| F3 | Carboxymethyl | 50 | mg/mL | 3.5 |
| | cellulose (CMC) | | | |
| | Sucrose | 90 | mg/mL | 62 |
| | Aluminum Phosphate | 0.25 | mg/mL | 0.18 |
| | Adjuvant (APA) | | | |
| | Polysorbate 20 | 2 | mg/mL | 1.4 |
| | Sodium Chloride | 150 | mM | 6.3 |
| | L-Histidine | 20 | mM | 2.2 |
| F4 | 2-Hydroxyethyl | 5 | mg/mL | 3.5 |
| | cellulose (2-HEC) | | | |
| | Sucrose | 90 | mg/mL | 62 |
| | Aluminum Phosphate | 0.25 | mg/mL | 0.18 |
| | Adjuvant (APA) | | | |
| | Polysorbate 20 | 2 | mg/mL | 1.4 |
| | Sodium Chloride | 150 | mM | 6.3 |
| | L-Histidine | 20 | mM | 2.2 |
| F5 | Hydroxypropyl | 5 | mg/mL | 3.5 |
| | cellulose (HPC) | | | |
| | Sucrose | 90 | mg/mL | 62 |
| | Aluminum Phosphate | 0.25 | mg/mL | 0.18 |
| | Adjuvant (APA) | | | |
| | Polysorbate 20 | 2 | mg/mL | 1.4 |
| | Sodium Chloride | 150 | mM | 6.3 |
| | L-Histidine | 20 | mM | 2.2 |
| F6 | PEG 400 | 10 | mg/mL | 7.0 |
| | Sucrose | 20 | mg/mL | 14 |
| | Aluminum Phosphate | 0.25 | mg/mL | 0.18 |
| | Adjuvant (APA) | | | |
| | Polysorbate 20 | 2 | mg/mL | 1.4 |
| | Sodium Chloride | 150 | mM | 6.3 |
| | L-Histidine | 20 | mM | 2.2 |
| F7 | PEO 100K | 10 | mg/mL | 7.0 |
| | Sucrose | 20 | mg/mL | 14.0 |
| | Aluminum Phosphate | 0.25 | mg/mL | 0.18 |
| | Adjuvant (APA) | | | |
| | Polysorbate 20 | 2 | mg/mL | 1.4 |
| | Sodium Chloride | 150 | mM | 6.3 |
| | L-Histidine | 20 | mM | 2.2 |
| F8 | Glycerol | 10 | mg/mL | 7.0 |
| | Sucrose | 20 | mg/mL | 14 |
| | Aluminum Phosphate | 0.25 | mg/mL | 0.18 |
| | Adjuvant (APA) | | | |
| | Polysorbate 20 | 2 | mg/mL | 1.4 |
| | Sodium Chloride | 150 | mM | 6.3 |
| | L-Histidine | 20 | mM | 2.2 |
| F9 | Propylene Glycol (PG) | 10 | mg/mL | 7.0 |
| | Sucrose | 20 | mg/mL | 14 |
| | Aluminum Phosphate | 0.25 | mg/mL | 0.18 |
| | Adjuvant (APA) | | | |
| | Polysorbate 20 | 2 | mg/mL | 1.4 |
| | Sodium Chloride | 150 | mM | 6.3 |
| | L-Histidine | 20 | mM | 2.2 |
| F10 | Carboxymethyl | 5 | mg/mL | 3.5 |
| | cellulose (CMC) | | | |
| | Propylene Glycol (PG) | 5 | mg/mL | 3.5 |
| | Sucrose | 90 | mg/mL | 63.14 |
| | Aluminum Phosphate | 0.25 | mg/mL | 0.18 |
| | Adjuvant (APA) | | | |
| | Polysorbate 20 | 2 | mg/mL | 1.4 |
| | Sodium Chloride | 150 | mM | 6.3 |
| | L-Histidine | 20 | mM | 2.2 |
| F11 | Carboxymethyl | 5 | mg/mL | 3.5 |
| | cellulose (CMC) | | | |
| | Sucrose | 40 | mg/mL | 28 |
| | Mannitol | 60 | mg/mL | 42 |
| | Aluminum Phosphate | 0.25 | mg/mL | 0.18 |
| | Adjuvant (APA) | | | |
| | Polysorbate 20 | 2 | mg/mL | 1.4 |
| | Sodium Chloride | 150 | mM | 6.3 |
| | L-Histidine | 20 | mM | 2.2 |
| F12 | Carboxymethyl | 5 | mg/mL | 3.5 |
| | cellulose (CMC) | | | |
| | Propylene Glycol (PG) | 5 | mg/mL | 3.5 |
| | Sucrose | 40 | mg/mL | 28 |
| | Mannitol | 60 | mg/mL | 42 |
| | Aluminum Phosphate | 0.25 | mg/mL | 0.18 |
| | Adjuvant (APA) | | | |
| | Polysorbate 20 | 2 | mg/mL | 1.4 |
| | Sodium Chloride | 50 | mM | 2.1 |
| | L-Histidine | 20 | mM | 2.2 |
| F13 | Aluminum Phosphate | 0.25 | mg/mL | 0.18 |
| | Adjuvant (APA) | | | |
| | Polysorbate 20 | 2 | mg/mL | 1.4 |
| | Sodium Chloride | 150 | mM | 6.3 |
| | L-Histidine | 20 | mM | 2.2 |

All excipient stock solutions were QS'ed volumetrically to 100 mL and then filtered through a 0.22 μm PES filtration unit (Millipore, Billerica, Mass.), and stored at ambient room temperature.

### Formulation and Filling of Adjuvanted Excipient Blend and PCV Vaccine

To prepare the vaccine formulation, first the aluminum adjuvant was combined with the excipient stock by adding concentrated sterile aluminum adjuvant to excipient stock, at volumetric ratios to obtain the final formulation target volume. The mixture was mixed to ensure homogeneity for 1 hour at 200 rpm, after which sterile filtered pneumococcal polysaccharide conjugates were added to the adjuvanted excipient blend, representing 25% of the final vaccine formulation volume. The vaccine formulation was further mixed for 1 hour at 200 rpm. The final vaccine formulations contained 64 μg pneumococcal polysaccharide/mL (8 μg/mL serotype 6B polysaccharide, 4 μg/mL polysaccharide for all other serotypes) (5.6 μg serotype 6B polysaccharide per vial, 2.8 μg polysaccharide per vial for all other serotypes) at pH 5.8. Formulations were then filled asceptically into sterile glass 2R vials at 0.7 mL. Table 2 describes the excipient components and total solids content for each of the individual thirteen formulations prepared.

TABLE 2
Final Vaccine Formulation Excipient & Solids
Content Percentages

| | | | Total Excipient |
|---|---|---|---|
| | | % | Solids Content |
| Key | Excipients | (w/v) | (% w/v) |
| Fl | Mannitol | 5.0 | 7.0 |
| | Sucrose | 2.0 | |
| F2 | Mannitol | 6.0 | 9.9 |
| | Sucrose | 4.0 | |
| F3 | Carboxymethyl cellulose (CMC) | 0.5 | 9.4 |
| | Sucrose | 8.9 | |
| F4 | 2-Hydroxyethyl cellulose (2-HEC) | 0.5 | 9.4 |
| | Sucrose | 8.9 | |
| F5 | Hydroxypropyl cellulose (HPC) | 0.5 | 9.4 |
| | Sucrose | 8.9 | |
| F6 | PEG 400 | 1.0 | 3.0 |
| | Sucrose | 2.0 | |
| F7 | PEO 100K | 1.0 | 3.0 |
| | Sucrose | 2.0 | |
| F8 | Glycerol | 1.0 | 3.0 |
| | Sucrose | 2.0 | |
| F9 | Propylene Glycol (PG) | 1.0 | 3.0 |
| | Sucrose | 2.0 | |
| F10 | Carboxymethyl cellulose (CMC) | 0.5 | 9.9 |
| | Propylene Glycol (PG) | 0.5 | |
| | Sucrose | 8.9 | |
| F11 | Carboxymethyl cellulose (CMC) | 0.5 | 10.4 |
| | Sucrose | 4.0 | |
| | Mannitol | 6.0 | |
| F12 | Carboxymethyl cellulose (CMC) | 0.5 | 10.9 |
| | Propylene Glycol (PG) | 0.5 | |
| | Sucrose | 4.0 | |
| | Mannitol | 6.0 | |
| F13 | Control formulation (no | 0 | na |
| | excipients added) | | |

### Process of Conjugate Adsorption to Aluminum Adjuvant

The process for adsorbing conjugates to aluminum adjuvant begins when concentrated aluminum adjuvant is first diluted in physiological saline (150-154 mM Sodium Chloride) to a desired volumetric ratio which results in a final adsorbed vaccine concentration of between 0.1 to 1.25 mg Al/mL of the aluminum adjuvant. The exact volume and mass of aluminum adjuvant diluted varies based upon the desired dose of adjuvant. In this example, a 50 mL final vaccine formulation was targeted; therefore 4.6 mL of concentrated aluminum adjuvant was added to 32.9 mL of saline to produce 37.5 mL of dilute aluminum adjuvant. The next step in the process involves mixing the dilute adjuvant for an hour, ensuring that a vortex is pulled, at 200 rpm when using a 1-2″ stir bar within a sterile glass vessel followed by slowly adding sterile filtered conjugates to the diluted aluminum adjuvant while maintaining 200 rpm constantly mixing, either singly if preparing a monovalent formulation, or adding as a polyvalent mixture if preparing a multi-valent vaccine formulation. In the example shown here, for a 50 mL final vaccine formulation, 12.5 mL of sterile polyvalent conjugate blend was added to 37.5 mL of dilute aluminum adjuvant. Following conjugate addition, the suspension was mixed for an additional hour of 200 rpm mixing as previously described. The formulated vaccine was then stored at 4° C. overnight (or >12 hours) to enable further adsorption of the conjugate to occur. Prior to dispense and use, all vaccine formulations are conditioned back to ambient laboratory temperature (21-25° C.), and well mixed for at least an hour.

### Formulation Storage and Freezing

Formulations were stored at 4° C. to serve as liquid controls. Frozen control samples were prepared by fully resuspending formulation following filling, and then immediately rapidly freezing by liquid nitrogen blast freezing at −115° C. Lyophilized and microwave dried formulations were initially blast frozen in the same manner as the frozen controls. Lyosphere formulations were removed from 4° C., resuspended and rapidly frozen by pipetting 100 uL on a cold plate chilled down to ≤−180° C. All vaccine formulations (prepared for vial/lyophilization, vial/REV drying, and lyosphere/lyophilization) were stored at −70° C. until the commencement of the subsequent drying process.

### Vaccine Lyophilization and Conventional Freeze Drying

Lyophilization was performed utilizing a Lyostar III (SP Scientific, Stone Ridge, N.Y.). All frozen vial formulations were loaded into the lyophilizer with a pre-cooled shelf at −50° C. Shelf temperature was ramped from −50° C. to −30° C. with a ramp rate of 0.1° C./min and primary drying was performed at −30° C./50 mTorr. For secondary drying, the shelf temperature was ramped to a 25° C. set point at 0.1° C./min ramp rate, and samples were held for 6 hours. Formulations containing mannitol were annealed prior to primary drying by raising the shelf temperature from −50° C. to −20° C. at 0.5° C./min, with a 180 minute hold, prior to returning to −50° C.

### Microwave Radiant Energy Vacuum (REV) Drying of Vaccine

Blast frozen vials were placed within a radiant energy vacuum (REV) dryer. Through REV drying frozen vaccine was dried through the combination of vacuum, pressure, and application of microwave energy in a travelling wave format. All formulations were loaded at −70° C. and immediately placed under 60-70 mTorr, after which 200W of radiant microwave energy was applied for nearly 7 hours, followed by 400W for 20 minutes.

### Generation of Vaccine Lyospheres and Conventional Freeze Drying

Lyospheres were generated by resuspending formulation by stirring for 1 hour at 200 rpm and then pipetting 100 uL of formulation onto a liquid nitrogen pre-cooled metal well plate. After the generation of frozen vaccine spheres, individual spheres were transferred within a biosafety cabinet into a separate container for each individual formulation. Lyospheres were then stored at −70° C. within a freezer until lyophilization. Lyosphere formulations were removed from −70° C. storage and placed into separate and distinct metal trays on a pre-cooled −50° C. shelf in a Lyostar III lyophilization chamber (SP Scientific, Stone Ridge, N.Y.).

All formulations were loaded and maintained at −50° C. prior to primary drying. For all formulations primary drying was performed at 15° C., under 30 mTorr, with a 0.4° C./min ramp rate, followed by secondary drying at a 30° C. set point, under 30 mTorr, at a 0.2° C./min ramp rate, and samples held for 6 hours. Formulations containing mannitol were annealed prior to primary drying by raising the shelf temperature from −50° C. to −20° C. at 0.5° C./min, holding for 60 minutes, then returning to −45° C. at 0.5° C./min and holding for 15 minutes, prior to returning to −50° C. for 30 minutes, prior to the initiation of primary drying.

### Reconstitution Time Analysis

Vaccine formulations which had been dried by conventional lyophilization, REV drying, or freeze-dried as lyospheres were removed from −70C storage. Individual formulations were reconstituted with 700 uL of sterile water. After reconstitution concentrations are similar to those prior to drying. Visual observations and the time for full reconstitution of the sample were recorded.

### Particle Size Analysis

The physical stability of the adjuvanted vaccine formulations with regard to aggregation was evaluated by measuring the particle size through the use of static light scattering (SLS). Samples were prepared in sterile-filtered and degassed physiological saline at a final analysis concentration of 14.5 μg Al/mL. Evaluation of the particle size and particle size distribution was performed using a Malvern© Mastersizer 2000 system, equipped with blue laser detection. In SLS analysis, the sample is recirculated through a transparent glass flow cell, allowing red and blue laser light to pass through. An array of large angle, back scatter, and focal plane detectors collect the multi-angle light scattering of the particles in solution, and a diffraction pattern is collected. The method relies on the principle that the diffraction angle is inversely proportional to the particle size. The scattering profile resulting from laser diffraction of all particles is analyzed using the application of Mie theory, which accounts for the influence of refractive index on light scattering behavior, relative particle transparencies, and extinction efficiencies of the particles. The resulting calculated particle diameter determined is a volume-based particle size measurement, and it is an average of three runs.

An explanation of the differences between the reported calculated diameter values reported by SLS analysis is described in Table 3. The D[4,3] value is relevant to report because it reflects the size of particles that make up a bulk of the sample volume. It is most sensitive to the presence of larger particulates in the particle size distribution. In addition, the D[3,2] value is relevant and most sensitive to the presence of smaller/fine particles in the particle size distribution. Further, the d(0.5) is relevant to report because it represents the maximum particle diameter below which 50% of the volume of the sample exists. The d(0.1) and d(0.9) values report the particle diameter, respectively, below which 10% or 90% of sample lies.

TABLE 3
Explanation of Particle Size Diameters Provided by Static
Light Scattering

| Description of | | Term | |
|---|---|---|---|
| Diameter Value | Detects | Utilized | Calculation |
| Volume-Weighted Mean (DeBrouckere) | Changes in the coarse (largest) particles | D[4, 3] | $D [ 4 , 3 ] = \frac{{\sum }_{\text{?}}^{\text{?}} D \text{?}}{{\sum }_{\text{?}}^{\text{?}} D \text{?}}$ |
| Surface Area- Weighted Mean (Sauter) | Changes in the fine (smallest) particles | D[3, 2] | $D [ 3 , 2 ] = \frac{{\sum }_{\text{?}}^{\text{?}} D \text{?}}{{\sum }_{\text{?}}^{\text{?}} D \text{?}}$ |
| Volume Median | 50% of | d(0.5) | Mid-point of |
| | distribution | | distribution |
| | is above this; | | |
| | 50% is | | |
| | below | | |

$\text{?} \text{indicates text missing or illegible when filed}$

### Results & Discussion

### 1) Particle Size and Distribution of Pneumococcal Conjugate Vaccine Lacking Additional Excipients

Table 4 reports the change in raw mean particle diameter of the vaccine formulation by comparing a non-frozen liquid control to −70° C. frozen, conventionally freeze-dried, REV-dried, and lyosphere formulations. The results illustrates the change in the vaccine particle size as a function of freeze-drying method. The volume weighted average particle size (D[4,3]) and the median particle size (d(0.5)) both increase in response to freeze-drying, from 8.3 μm to 10.0, 12.2, and 15.0 μm with lyosphere, REV-drying, and conventional lyophilization respectively. Each of the freeze-drying methods mitigate the degree of agglomeration observed when vaccine is frozen, which otherwise results in a particle size of 78.2 μm. However, the base formulation without excipients still agglomerates upon freeze-drying, relative to the liquid control. For this reason, new formulations containing excipients amenable for freeze-drying should be theoretically beneficial to preventing or reducing this increase.

TABLE 4
Raw SLS Data of Freeze-Dried Pneumoccal Conjugate Vaccine

| | D [4, 3] | d (0.5) | D [3, 2] |
|---|---|---|---|
| Sample Name | μm | μm | μm |
| F13 4° C. LIQUID | 8.3 | 7.8 | 7.1 |
| F13 FROZEN −70° C. | 78.2 | 60.2 | 33.2 |
| F13 LYOPHILIZED | 15.0 | 11.5 | 9.0 |
| F13 REV-DRIED | 12.2 | 9.3 | 7.7 |
| F13 LYOSPHERE | 10.0 | 6.8 | 5.2 |

### 2) Key Excipient Combinations Improve the Particle Size and Distribution of Frozen Pneumococcal Conjugate Vaccine

The mean particle size of frozen vaccine, decreases when combinations of particular excipients are utilized. Freezing PCV in the presence of 5% w/v mannitol/2% w/v sucrose (F1), or 6% w/v mannitol/4% w/v sucrose (F2), results in a significant reduction in the freeze-agglomeration; from 78.2 μm down to 12.1 μm (F1) and 15.3 μm (F2). Further, freezing formulations 3,10, 11, and 12 results in smaller average particle size than even the control formulation, between 5.2 to 5.9 m, Table 5. These results suggest all formulations tested improve the particle size over the frozen, non-excipient containing PCV formulation (F13). Formulations 1, 2, 4, 5, 6, 7, 8, and all reduce the extent of freeze-induced agglomeration of the vaccine. Formulations 3, 10, 11, and 12, however, further reduce the degree of agglomeration and improve upon the base formulation particle size, following −70° C. freezing.

TABLE 5
Raw SLS Data of Pneumoccal Conjugate Vaccine
Formulations Resistant to Freeze Agglomeration

| | | | | Improved | Improved |
|---|---|---|---|---|---|
| | D | d | D | Over | Over |
| | [4, 3] | (0.5) | [3, 2] | Frozen | Liquid |
| Sample Name | μm | μm | μm | Control | Control |
| F13 4° C. LIQUID | 8.3 | 7.8 | 7.1 | + | − |
| F13 FROZEN | 78.2 | 60.2 | 33.2 | − | − |
| −70° C. | | | | | |
| F4 FROZEN | 22.2 | 10.4 | 9.0 | + | − |
| −70° C. | | | | | |
| F8 FROZEN | 18.1 | 15.2 | 12.1 | + | − |
| −70° C. | | | | | |
| F2 FROZEN | 15.3 | 12.5 | 10.0 | + | − |
| −70° C. | | | | | |
| F9 FROZEN | 14.8 | 10.9 | 9.4 | + | − |
| −70° C. | | | | | |
| F7 FROZEN | 13.8 | 10.0 | 8.7 | + | − |
| −70° C. | | | | | |
| F6 FROZEN | 13.0 | 9.7 | 8.5 | + | − |
| −70° C. | | | | | |
| F1 FROZEN | 12.1 | 10.5 | 8.5 | + | − |
| −70° C. | | | | | |
| F5 FROZEN | 11.5 | 8.5 | 7.7 | + | − |
| −70° C. | | | | | |
| F3 FROZEN | 5.9 | 5.4 | 4.4 | + | + |
| −70° C. | | | | | |
| F10 FROZEN | 5.8 | 5.3 | 4.4 | + | + |
| −70° C. | | | | | |
| F12 FROZEN | 5.6 | 5.1 | 4.3 | + | + |
| −70° C. | | | | | |
| F11 FROZEN | 5.2 | 4.7 | 3.9 | + | + |
| −70° C. | | | | | |

### 3) Key Excipient Combinations Improve the Particle Size and Distribution of Lyophilized Pneumococcal Conjugate Vaccine

The process of lyophilization increases the PCV vaccine mean particle size, from 8.3 μm to 15 μm. Utilizing formulations 5 and 4 results in post-lyophilization sizes similar to the liquid control, between 8.0 μm and 8.8 μm, Table 6. Four formulations reduce the particle size below that of the 4C liquid control, namely F10, F3, F12, and F11, spanning 5.4 μm to 7.3 μm mean particle size, Table 6. The results indicate that formulations 4 and 5 prevent the increase in particle size observed after lyophilization. Further, formulations 3, 10, 11, and 12 not only prevent the increase observed, but also control and reduce the particle size of the vaccine well below that of the liquid control.

TABLE 6
Raw SLS Data of Pneumoccal Conjugate Vaccine Formulations
Resistant to Lyophilization-Induced Agglomeration

| | | | | Improved | Improved |
|---|---|---|---|---|---|
| | D | d | D | Over | Over |
| | [4, 3] | (0.5) | [3, 2] | Lyophilized | Liquid |
| Sample Name | μm | μm | μm | Control | Control |
| F13 4° C. | 8.3 | 7.8 | 7.1 | + | − |
| LIQUID | | | | | |
| CONTROL | | | | | |
| F13 | 15.0 | 11.5 | 9.0 | − | − |
| LYOPHILIZED | | | | | |
| CONTROL | | | | | |
| F5 | 8.8 | 8.0 | 7.1 | + | − |
| LYOPHILIZED | | | | | |
| F4 | 8.0 | 7.2 | 6.4 | + | + |
| LYOPHILIZED | | | | | |
| F11 | 7.3 | 5.5 | 4.5 | + | + |
| LYOPHILIZED | | | | | |
| F12 | 5.7 | 5.2 | 4.3 | + | + |
| LYOPHILIZED | | | | | |
| F3 | 5.5 | 5.1 | 4.3 | + | + |
| LYOPHILIZED | | | | | |
| F10 | 5.4 | 4.9 | 4.2 | + | + |
| LYOPHILIZED | | | | | |

Two formulations in particular increased the degree of lyophilization induced agglomeration, namely F1 and F2, containing 2% w/v mannitol 5% w/v sucrose or 4% w/v mannitol/6% w/v sucrose, respectively. The resulting particle size and heterogeneity increased from 15 μm post lyophilization to 26.2 μm and 28.2 μm for each of the vaccine formulations, Table 7. Formulations 6, 7, 8, and 9 did not produce lyo cakes and were also not moved forward for further evaluation by REV drying or lyospheres.

TABLE 7
Raw SLS Data of Pneumoccal Conjugate Vaccine Formulations
Agglomerated by Lyophilization

| | | | | Improved | Improved |
|---|---|---|---|---|---|
| | D | d | D | Over | Over |
| | [4, 3] | (0.5) | [3, 2] | Lyophilized | Liquid |
| Sample Name | μm | μm | μm | Control | Control |
| F13 4° C. | 8.3 | 7.8 | 7.1 | + | − |
| LIQUID | | | | | |
| CONTROL | | | | | |
| F13 | 15.0 | 11.5 | 9.0 | − | − |
| LYOPHILIZED | | | | | |
| CONTROL | | | | | |
| F2 | 28.2 | 23.5 | 14.1 | − − | − − |
| LYOPHILIZED | | | | | |
| F1 | 26.2 | 21.2 | 12.9 | − − | − − |
| LYOPHILIZED | | | | | |

| | | | |
|---|---|---|---|
| F6 | Cake Collapse | − − | − − |
| LYOPHILIZED | | | |
| F7 | Cake Collapse | − − | − − |
| LYOPHILIZED | | | |
| F8 | Cake Collapse | − − | − − |
| LYOPHILIZED | | | |
| F9 | Cake Collapse | − − | − − |
| LYOPHILIZED | | | |

### 4) Key Excipient Combinations Improve the Particle Size and Distribution of REV-Dried Pneumococcal Conjugate Vaccine

The particle size of the control vaccine formulation increases from 8.3 μm to 12.2 μm upon REV drying. Formulation 2 containing 4% w/v mannitol/6% w/v sucrose did not significantly alter or reduce the degree of agglomeration observed due to REV drying, producing a 12.0 μm mean particle size, Table 8.

In contrast, two formulations, F4 and decreased the REV induced agglomeration to 9.1 μm and 8.6 μm, respectively. Five specific formulations, 3, 5, 10, 11, and 12 reduced the mean particle size below that of the liquid control, from 4.7 μm up to 7.7 lam, relative to the liquid control at 8.3 m. As mentioned previously, formulations 6, 7, 8, and 9 were not tested.

TABLE 8
Raw SLS Data of Pneumoccal Conjugate Vaccine Formulations
Resistant to REV Agglomeration

| | | | | Improved | Improved |
|---|---|---|---|---|---|
| | D | d | D | Over | Over |
| | [4, 3] | (0.5) | [3, 2] | REV | Liquid |
| Sample Name | μm | μm | μm | Control | Control |
| F13 4° C. | 8.3 | 7.8 | 7.1 | + | − |
| LIQUID | | | | | |
| CONTROL | | | | | |
| F13 REV | 12.2 | 9.3 | 7.7 | − | − |
| CONTROL | | | | | |
| F2 REV-DRIED | 12.0 | 8.7 | 6.8 | + | − |
| F4 REV-DRIED | 9.1 | 7.3 | 6.2 | + | − |
| F1 REV-DRIED | 8.6 | 7.3 | 5.8 | + | − |
| F5 REV-DRIED | 7.7 | 6.8 | 6.1 | + | + |
| F3 REV-DRIED | 5.3 | 4.8 | 3.7 | + | + |
| F10 REV-DRIED | 5.2 | 4.7 | 3.8 | + | + |
| F12 REV-DRIED | 5.0 | 4.6 | 3.8 | + | + |
| F11 REV-DRIED | 4.7 | 4.2 | 3.5 | + | + |

| | | | |
|---|---|---|---|
| F6 REV-DRIED | Not tested. | − | − |
| F7 REV-DRIED | Not tested. | − | − |
| F8 REV-DRIED | Not tested. | − | − |
| F9 REV-DRIED | Not tested. | − | − |

### 5) Key Excipient Combinations Improve the Particle Size and Distribution of Lyosphere Pneumococcal Conjugate Vaccine

As originally illustrated in Table 4, the production of lyospheres results in the least degree of agglomeration relative to the other drying methods evaluated. Upon lyosphere formation, the control vaccine particle size increases from 8.3 μm to 10.0 μm. Formulation 1 slightly reduces the mean particle size from 10.0 μm to 9.1 μm, Table 9. Formulations 2, 3, 4, 5, 12, 3, 1, and 11 all reduce both the particle size and agglomeration below the 8.3 μm liquid control to between 4.4 μm and 7.9 μm. Most notably are formulations 3, 10, 11, and 12 which produce a reduced particle size from 8.3 μm down to 4.4-5.5 μm.

TABLE 9
Raw SLS Data of Pneumoccal Conjugate Vaccine Formulations
Resistant to Lyosphere Agglomeration

| | | | | Improved | Improved |
|---|---|---|---|---|---|
| | D | d | D | Over | Over |
| | [4, 3] | (0.5) | [3, 2] | Lyosphere | Liquid |
| Sample Name | μm | μm | μm | Control | Control |
| F13 4° C. | 8.3 | 7.8 | 7.1 | + | − |
| LIQUID | | | | | |
| CONTROL | | | | | |
| F13 | 10.0 | 6.8 | 5.2 | − | − |
| LYOSPHERE | | | | | |
| CONTROL | | | | | |
| F1 | 9.1 | 7.5 | 6.0 | + | − |
| LYOSPHERE | | | | | |
| F2 | 7.9 | 7.2 | 5.6 | + | + |
| LYOSPHERE | | | | | |
| F4 | 7.0 | 6.4 | 5.7 | + | + |
| LYOSPHERE | | | | | |
| F5 | 6.6 | 6.1 | 4.8 | + | + |
| LYOSPHERE | | | | | |
| F12 | 5.5 | 4.1 | 3.4 | + | + |
| LYOSPHERE | | | | | |
| F3 | 4.8 | 4.2 | 3.4 | + | + |
| LYOSPHERE | | | | | |
| F10 | 4.4 | 3.8 | 3.2 | + | + |
| LYOSPHERE | | | | | |
| F11 | 4.4 | 3.7 | 3.0 | + | + |
| LYOSPHERE | | | | | |

| | | | |
|---|---|---|---|
| F6 | Not tested. | − − | − − |
| LYOSPHERE | | | |
| F7 | Not tested. | − − | − − |
| LYOSPHERE | | | |
| F8 | Not tested. | − − | − − |
| LYOSPHERE | | | |
| F9 | Not tested. | − − | − − |
| LYOSPHERE | | | |

### 6) Key Excipient Combinations Improve the Particle Size and Distribution of Liquid Pneumococcal Conjugate Vaccine

Particular excipients maintain or improve the particle size of the liquid PCV formulation, while a select few lead to aggregation. The particle size of liquid PCV increases significantly from 8.3 μm to 75.4 μm, 36.7 μm, 34.7 μm, and 24.2 μm, when prepared as formulations 10, 3, 2, and 4 respectively, Table 10. In contrast, formulations 8 and 9 maintain the particle size between 8.3-8.4 μm. Further improvement in the liquid vaccine particle size is observed for formulations 1, 5, 6, 7, 11, and 12; ranging from 6.1-7.7 μm, below that of the control formulation.

Finally, Table 11 below summarizes the performance of the formulations, presentation, and drying methods relative to one another. Liquid, Frozen, Lyophilization, REV drying, and Lyosphere samples were tested using SLS and the results compared and relative performance reported on in table 11. As can be observed, particular formulations perform well across all presentations; specifically formulations 11 and 12, however, if accounting for freeze-drying process size only, then formulations 3, 10, 11, and 12 outperform all others evaluated. Further, particular formulations perform most optimally in particular freeze-drying applications.

TABLE 10
Raw SLS Data of Liquid Pneumoccal Conjugate Vaccine
Formulations

| Sample Name | D [4, 3] | d (0.5) | D [3, 2] |
|---|---|---|---|
| F13 4° C. LIQUID | 8.3 | 7.8 | 7.1 |
| F10 4° C. LIQUID | 75.4 | 6.1 | 4.9 |
| F3 4° C. LIQUID | 36.7 | 6.1 | 4.7 |
| F2 4° C. LIQUID | 34.7 | 7.4 | 6.5 |
| F4 4° C. LIQUID | 24.2 | 6.6 | 5.6 |
| F8 4° C. LIQUID | 8.4 | 7.8 | 7.1 |
| F9 4° C. LIQUID | 8.3 | 7.8 | 7.3 |
| F7 4° C. LIQUID | 7.7 | 7.1 | 6.5 |
| F1 4° C. LIQUID | 7.5 | 6.9 | 6.3 |
| F6 4° C. LIQUID | 6.9 | 6.5 | 6.0 |
| F5 4° C. LIQUID | 6.9 | 6.5 | 5.6 |
| F12 4° C. LIQUID | 6.2 | 5.7 | 4.5 |
| F11 4° C. LIQUID | 6.1 | 5.6 | 4.6 |

TABLE 11
Performance Ranking of PCV Formulations

| Key | Liquid | Frozen | Lyophilization | Lyosphere | REV |
|---|---|---|---|---|---|
| F1 | +++ | ++ | − | +++ | +++ |
| F2 | − | + | − | +++ | ++ |
| F3 | − | +++ | +++ | +++ | +++ |
| F4 | + | + | +++ | +++ | +++ |
| F5 | +++ | ++ | +++ | +++ | +++ |
| F6 | +++ | ++ | n/a | n/a | n/a |
| F7 | +++ | ++ | n/a | n/a | n/a |
| F8 | +++ | + | n/a | n/a | n/a |
| F9 | +++ | ++ | n/a | n/a | n/a |
| F10 | − | +++ | +++ | +++ | +++ |
| F11 | +++ | +++ | +++ | +++ | +++ |
| F12 | ++ | +++ | +++ | +++ | +++ |
| F13 | Ref | − | ++ | +++ | ++ |
| | (+++) | | | | |

Key:
[+++] (0.5 and 4, 3) ≤ 10 μm, [++] d(0.5 and 4, 3) ≤ 15 μm, [+] d(0.5 and 4, 3) ≤ 25 μm, [−] aggregated

1. A formulation comprising (i) one or more polysaccharide-protein conjugates; (ii) a buffer having a pH in the range from about 5.0 to 7.5; (ii) an alkali or alkaline salt selected from the group consisting of magnesium chloride, calcium chloride, potassium chloride, sodium chloride or a combination thereof, (iii) a surfactant; (iv) a sugar selected from the group consisting of sucrose, trehalose and raffinose; optionally (v) a bulking agent; and optionally (vi) a polymer selected from the group consisting of carboxymethyl cellulose (CMC), hydroxypropyl cellulose (HPC), hydroxypropyl methylcellulose (HPMC), 2-hydroxyethyl cellulose (2-HEC), crosscarmellose, methyl cellulose, glycerol, polyethylene oxide, polyethylene glycol (PEG) and propylene glycol (PG), or a combination thereof, and (vii) an aluminum adjuvant.

2. The formulation of claim 1, wherein the formulation comprises a bulking agent and wherein the total concentration of sugar and bulking agent is at least about 50 mg/ml.

3. The formulation of claim 1, wherein the formulation comprises a bulking agent and wherein the total concentration of sugar and bulking agent is at least about 90 mg/ml.

4. The formulation of claim 1, wherein the formulation comprises a bulking agent and the total concentration of sugar and bulking agent is about 50-400 mg/ml, and the bulking agent to sugar ratio is greater than or equal to 1.

5. The formulation of claim 1, wherein the formulation comprises a bulking agent and wherein the total concentration of sugar and bulking agent is about 50-150 mg/ml, and the bulking agent to sugar ratio is about 2:1.

6. The formulation of claim 1, wherein the formulation comprises a bulking agent that is mannitol, glycine or lactose.

7. The formulation of claim 1, wherein the sugar is trehalose or sucrose.

8. The formulation of claim 1, wherein the formulation comprises a polymer that is carboxymethyl cellulose (CMC), hydroxypropyl cellulose (HPC), 2-hydroxyethyl cellulose (2-HEC), glycerol, polyethylene oxide, polyethylene glycol (PEG) or propylene glycol (PG) at about 1-25 mg/ml, or a combination thereof.

9. The formulation of claim 1, wherein the polymer is carboxymethyl cellulose (CMC), hydroxypropyl cellulose (HPC), 2-hydroxyethyl cellulose (2-HEC) at about 1-10 mg/ml, or a combination thereof.

10. The formulation of claim 1, wherein the surfactant is polysorbate or a poloxamer having a molecular weight in the range from 1100 Da to 17,400 Da.

11. The formulation of claim 10, wherein the poloxamer has a molecular weight in the range from 7,500 Da to 15,000 Da.

12. The formulation of claim 10, wherein the poloxamer is poloxamer 188 or poloxamer 407.

13. The formulation of claim 10, wherein the final concentration of the poloxamer is from about 0.01 to 50 mg/ml.

14. The formulation of claim 10, wherein the final concentration of the poloxamer is from about 0.25 to 10 mg/ml.

15. The formulation of claim 1, wherein the surfactant is polysorbate 20 or 80.

16. The formulation of claim 15, wherein the final concentration of the polysorbate 20 is in the range from about 0.01 to 100 mg/ml.

17. The formulation of claim 15, wherein the final concentration of the polysorbate 20 is in the range from about 0.25 to 25 mg/ml.

18. The formulation of claim 15, wherein the final concentration of the polysorbate 20 is in the range from about 1 to 5 mg/ml.

19. The formulation of claim 1 to, wherein the pH buffer has a pH in the range from about 5.0 to 7.0.

20. The formulation of claim 19, wherein the buffer is selected from the group consisting of phosphate, succinate, histidine, MES, MOPS, HEPES, acetate and citrate.

21. The formulation of claim 20, wherein the buffer is histidine at a final concentration of about 5 mM to 50 mM, or succinate at a final concentration of about 1 mM to 10 mM.

22. The formulation of claim 21, wherein the histidine is at a final concentration of about 20 mM.

23. The formulation of claim 1, wherein the salt is sodium chloride.

24. The formulation of claim 23, wherein the sodium chloride is present at a concentration from about 20 mM to 170 mM.

25. The formulation of claim 1, wherein the total polysaccharide-protein concentration is 2-704 μg/ml.

26. The formulation of claim 1, wherein the total polysaccharide-protein concentration is 4-92 μg/ml.

27. The formulation of claim 1 that comprises 0.1-0.5 mg/mL of Aluminum Phosphate Adjuvant (APA).

28. The formulation of claim 1, wherein the polysaccharide-protein conjugates comprise one or more pneumococcal polysaccharides conjugated to a carrier protein.

29. The formulation of claim 28, wherein the carrier protein is selected from CRM₁₉₇, diphtheria toxin fragment B (DTFB), DTFB C8, Diphtheria toxoid (DT), tetanus toxoid (TT), fragment C of TT, pertussis toxoid, cholera toxoid, meningococcal outer membrane protein complex (OMPC), E. coli LT, E. coli ST, exotoxin A from Pseudomonas aeruginosa, Protein D from Non-Typeable Haemophilus influenzae and combinations thereof.

30. The formulation of claim 28, wherein one or more of the polysaccharide-protein conjugates are conjugated to CRM₁₉₇.

31. The formulation of claim 28, wherein one or more of the polysaccharide-protein conjugates comprises capsular polysaccharides from at least one of serotypes 1, 2, 3, 4, 5, 6A, 6B, 6C, 6D, 6E, 6G, 6H, 7F, 7A, 7B, 7C, 8, 9A, 9L, 9N, 9V, 10F, 10A, 10B, 10C, 11F, 11A, 11B, 11C, 11D, 11E, 12F, 12A, 12B, 13, 14, 15F, 15A, 15B, 15C, 16F, 16A, 17F, 17A, 18F, 18A, 18B, 18C, 19F, 19A, 19B, 19C, 20A, 20B, 21, 22F, 22A, 23F, 23A, 23B, 24F, 24A, 24B, 25F, 25A, 27, 28F, 28A, 29, 31, 32F, 32A, 33F, 33A, 33B, 33C, 33D, 33E, 34, 35F, 35A, 35B, 35C, 36, 37, 38, 39, 40, 41F, 41A, 42, 43, 44, 45, 46, 47F, 47A, 48, CWPS1, CWPS2, CWPS3 of Streptococcus pneumoniae conjugated to one or more carrier proteins.

32. The formulation of claim 1, wherein the polysaccharide-protein conjugate formulation is a 15-valent pneumococcal conjugate (15vPnC) formulation consisting essentially of S. pneumoniae polysaccharide from serotypes 1, 3, 4, 5, 6A, 6B, 7F, 9V, 14, 18C, 19A, 19F, 22F, 23 F and 33F conjugated to CRM₁₉₇.

33. The formulation of claim 1, wherein one or more of the polysaccharide protein conjugates are prepared using reductive amination under DMSO conditions.

34. The formulation of claim 33, wherein the polysaccharide protein conjugates from serotypes 6A, 6B, 7F, 18C, 19A, 19F, and 23F are prepared under DMSO conditions and polysaccharide protein conjugates from serotypes 1, 3, 4, 5, 9V, 14, 22F, and 33F are prepared using aqueous conditions.

35. The formulation of claim 34, wherein each dose is formulated to contain: 4 μg/mL or 8 μg/mL of each saccharide, except for 6B at 8 μg/mL or 16 μg/mL; and about 64 μg/mL or 128 μg/mL CRM₁₉₇ carrier protein.

36. The formulation of claim 1 that comprises a pH buffered saline solution having a pH in the range from about 5.0 to 7.5, about 150 mM NaCl, about 2 mg/ml Polysorbate 20, about 50 mg/ml mannitol, and about 20 mg/ml sucrose.

37. The formulation of claim 1 that comprises a pH buffered saline solution having a pH in the range from about 5.0 to 7.5, about 150 mM NaCl, about 2 mg/ml Polysorbate 20, about 60 mg/ml mannitol, about 40 mg/ml sucrose.

38. The formulation of claim 1 that comprises a pH buffered saline solution having a pH in the range from about 5.0 to 7.5, about 150 mM NaCl, about 2 mg/ml Polysorbate 20, about 90 mg/ml sucrose, about 5 mg/ml CMC.

39. The formulation of claim 1 that comprises a pH buffered saline solution having a pH in the range from about 5.0 to 7.5, about 150 mM NaCl, about 2 mg/ml Polysorbate 20, about 90 mg/ml sucrose, about 5 mg/ml 2-HEC.

40. The formulation of claim 1 that comprises a pH buffered saline solution having a pH in the range from about 5.0 to 7.5, about 150 mM NaCl, about 2 mg/ml Polysorbate 20, about 90 mg/ml sucrose, about 5 mg/ml HPC.

41. The formulation of claim 1 that comprises a pH buffered saline solution having a pH in the range from about 5.0 to 7.5, about 150 mM NaCl, about 2 mg/ml Polysorbate 20, about 90 mg/ml sucrose, about 5 mg/ml CMC, and about 5 mg/ml PG.

42. The formulation of claim 1 that comprises a pH buffered saline solution having a pH in the range from about 5.0 to 7.5, about 150 mM NaCl, about 2 mg/ml Polysorbate 20, about 40 mg/ml sucrose, about 60 mg/ml mannitol, and about 5 mg/ml CMC.

43. The formulation of claim 1 that comprises a pH buffered saline solution having a pH in the range from about 5.0 to 7.5, about 150 mM NaCl, about 2 mg/ml Polysorbate 20, about 40 mg/ml sucrose, about 60 mg/ml mannitol, about 5 mg/ml CMC and about 5 mg/ml PG.

44. The formulation of claim 1 that comprises a 15-valent pneumococcal conjugate (15vPnC) consisting essentially of S. pneumoniae polysaccharide from serotypes 1, 3, 4, 5, 6A, 6B, 7F, 9V, 14, 18C, 19A, 19F, 22F, 23 F and 33F conjugated to CRM₁₉₇ at about 4-92 μg/ml, a pH buffered saline solution having a pH in the range from about 5.0 to 7.5, about 30-150 mM NaCl, about 0.05-2 mg/ml Polysorbate 20, about 20-250 mg/ml sucrose, about 30-100 mg/ml mannitol, about 0.1-0.75 mg/ml APA, about 1-10 mg/ml CMC and optionally about 1-10 mg/ml PG.

45. The formulation of claim 1 that comprises capsular polysaccharides from at least one of serotypes 1, 2, 3, 4, 5, 6A, 6B, 6C, 6D, 6E, 6G, 6H, 7F, 7A, 7B, 7C, 8, 9A, 9L, 9N, 9V, 10F, 10A, 10B, 10C, 11F, 11A, 11B, 11C, 11D, 11E, 12F, 12A, 12B, 13, 14, 15F, 15A, 15B, 15C, 16F, 16A, 17F, 17A, 18F, 18A, 18B, 18C, 19F, 19A, 19B, 19C, 20A, 20B, 21, 22F, 22A, 23F, 23A, 23B, 24F, 24A, 24B, 25F, 25A, 27, 28F, 28A, 29, 31, 32F, 32A, 33F, 33A, 33B, 33C, 33D, 33E, 34, 35F, 35A, 35B, 35C, 36, 37, 38, 39, 40, 41F, 41A, 42, 43, 44, 45, 46, 47F, 47A, 48, CWPS1, CWPS2, CWPS3 of Streptococcus pneumoniae conjugated to CRM₁₉₇ at about 4-92 μg/ml, a pH buffered saline solution having a pH in the range from about 5.0 to 7.5, about 30-150 mM NaCl, about 0.05-2 mg/ml Polysorbate 20, about 20-250 mg/ml sucrose, about 30-100 mg/ml mannitol, about 0.1-0.75 mg/ml APA, about 1-10 mg/ml CMC and optionally about 1-10 mg/ml PG.

46. The formulation of claim 1 that comprises capsular polysaccharides from at least one of serotypes 1, 2, 3, 4, 5, 6A, 6B, 6C, 6D, 6E, 6G, 6H, 7F, 7A, 7B, 7C, 8, 9A, 9L, 9N, 9V, 10F, 10A, 10B, 10C, 11F, 11A, 11B, 11C, 11D, 11E, 12F, 12A, 12B, 13, 14, 15F, 15A, 15B, 15C, 16F, 16A, 17F, 17A, 18F, 18A, 18B, 18C, 19F, 19A, 19B, 19C, 20A, 20B, 21, 22F, 22A, 23F, 23A, 23B, 24F, 24A, 24B, 25F, 25A, 27, 28F, 28A, 29, 31, 32F, 32A, 33F, 33A, 33B, 33C, 33D, 33E, 34, 35F, 35A, 35B, 35C, 36, 37, 38, 39, 40, 41F, 41A, 42, 43, 44, 45, 46, 47F, 47A, 48, CWPS1, CWPS2, CWPS3 of Streptococcus pneumoniae conjugated to CRM₁₉₇ at about 4-92 μg/ml, a buffer having a pH in the range from about 5.0 to 7.5; (ii) an alkali or alkaline salt selected from the group consisting of magnesium chloride, calcium chloride, potassium chloride, sodium chloride or a combination thereof at 20-170 mM; (iii) a surfactant that is polysorbate 20 at 1 to 5 mg/ml; (iv) a sugar selected from the group consisting of sucrose, trehalose and raffinose; optionally (v) a bulking agent that is mannitol; and optionally (vi) a polymer selected from the group consisting of carboxymethyl cellulose (CMC), hydroxypropyl cellulose (HPC), hydroxypropyl methylcellulose (HPMC), 2-hydroxyethyl cellulose (2-HEC) and Propylene Glycol (PG), or a combination thereof at 1-25 mg/ml; wherein the total concentration of sugar, or sugar and bulking agent is about 50-400 mg/ml.

47. The formulation of claim 1 that is an aqueous solution prior to lyophilization or microwave energy drying.

48. The formulation of claim 1 that is a reconstituted formulation in solution.

49. The formulation of claim 1 that has d(0.50) less than 15 μm.

50. The formulation of claim 1 that is in lyosphere form.
